SEQUENCE LISTING (15 sequences)

SEQ ID NO: 1 - protein, 263 aa, Escherichia coli
HPETLVKVKDAEDQLGARVGYIELDLNSGKILESFRPEERFPMVSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERDRQIAEIGASLIKHW

SEQ ID NO: 2 - protein, 263 aa, Escherichia coli
QTSAVQQKLAALEKSSGGRLGVALIDTADNTQVLYRGDERFPMCSTSKVMAAAAVLKQSETQKQLLNQPVEIKPADLVNYNPIAEKHVNGTMTLAELSAAALQYSDNTAMNKLIAQLGGPGGVTAFARAIGDETFRLDRTEPTLNTAIPGDPRDTTTPRAMAQTLRQLTLGHALGETQRAQLVTWLKGNTTGAASIRAGLPTSWTAGDKTGSGGYGTTNDIAVIWPQGRAPLVLVTYFTQPQQNAESRRDVLASAARIIAEGL

SEQ ID NO: 3 - protein, 257 aa, Staphylococcus aureus
KELNDLEKKYNAHIGVYALDTKSGKEVKFNSDKRFAYASTSKAINSAILLEQVPYNKLNKKVHINKDDIVAYSPILEKYVGKDITLKALIEASMTYSDNTANNKIIKEIGGIKKVKQRLKELGDKVTNPVRYEIELNYYSPKSKKDTSTPAAFGKTLNKLIANGKLSKENKKFLLDLMLNNKSGDTLIKDGVPKDYKVADKSGQAITYASRNDVAFVYPKGQSEPIVLVIFTNKDNKSDKPNDKLISETAKSVMKEF

SEQ ID NO: 4 - protein, 199 aa, Escherichia coli
MSNAKTKLGITKYSIVTNSNDSVTLRLMTEHDLAMLYEWLNRSHIVEWWGGEEARPTLADVQEQYLPSVLAQESVTPYIAMLNGEPIGYAQSYVALGSGDGRWEEETDPGVRGIDQLLANASQLGKGLGTKLVRALVELLFNDPEVTKIQTDPSPSNLRAIRCYEKAGFERQGTVTTPYGPAVYMVQTRQAFERTRSDA

SEQ ID NO: 5 - protein, 419 aa, Escherichia coli
MRFEEWVKDKHIPFKLNHPDDNYDDFKPLRKIIGDTRVVALGENSHFIKEFFLLRHTLLRFFIEDLGFTTFAFEFGFAEGQIINNWIHGQGTDDEIGRFLKHFYYPEELKTTFLWLREYNKAAKEKITFLGIDIPRNGGSYLPNMEIVHDFFRTADKEALHIIDDAFNIAKKIDYFSTSQAALNLHELTDSEKCRLTSQLARVKVRLEAMAPIHIEKYGIDKYETILHYANGMIYLDYNIQAMSGFISGGGMQGDMGAKDKYMADSVLWHLKNPQSEQKVIVVAHNAHIQKTPILYDGFLSCLPMGQRLKNAIGDDYMSLGITSYSGHTAALYPEVDTKYGFRVDNFQLQEPNEGSVEKAISGCGVTNSFVFFRNIPEDLQSIPNMIRFDSIYMKAELEKAFDGIFQIEKSSVSEVVYE

SEQ ID NO: 6 - protein, 388 aa, Bacteroides fragilis
MTMRIDTDKQMNLLSDKNVAIIGGGPVGLTMAKLLQQNGIDVSVYERDNDREARIFGGTLDLHKGSGQEAMKKAGLLQTYYDLALPMGVNIADKKGNILSTKNVKPENRFDNPEINRNDLRAILLNSLENDTVIWDRKLVMLEPGKKKWTLTFENKPSETADLVILANGGMSKVRKFVTDTEVEETGTFNIQADIHQPEINCPGFFQLCNGNRLMASHQGNLLFANPNNNGALHFGISFKTPDEWKNQTQVDFQNRNSVVDFLLKEFSDWDERYKELIHTTLSFVGLATRIFPLEKPWKSKRPLPITMIGDAAHLMPPFAGQGVNSGLVDALILSDNLADGKFNSIEEAVKNYEQQMFMYGKEAQEESTQNEIEMFKPDFTFQQLLNV

SEQ ID NO: 7 - protein, 267 aa, Enterococcus faecium
MLKQKELIANVKNLTESDERITACMMYGSFTKGEGDQYSDIEFYIFLKHSITSNFDSSNWLFDVAPYLMLYKNEYGTEVVIFDNLIRGEFHFLSEKDMNIIPSFKDSGYIPDTKAMLIYDETGQLENYLSEISGARPNRLTEENANFLLCNFSNLWLMGINVLKRGEYARSLELLSQLQKNTLQLIRMAEKNADNWLNMSKNLEKEISLENYKKFAKTTARLDKVELFEAYKNSLLLVMDLQSHLIEQYNLKVTHDILERLLNYISE

SEQ ID NO: 8 - protein, 532 aa, Artificial Sequence
  TEM36-GGGGGG-CTXM16
HPETLVKVKDAEDQLGARVGYIELDLNSGKILESFRPEERFPMVSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERDRQIAEIGASLIKHWGGGGGGQTSAVQQKLAALEKSSGGRLGVALIDTADNTQVLYRGDERFPMCSTSKVMAAAAVLKQSETQKQLLNQPVEIKPADLVNYNPIAEKHVNGTMTLAELSAAALQYSDNTAMNKLIAQLGGPGGVTAFARAIGDETFRLDRTEPTLNTAIPGDPRDTTTPRAMAQTLRQLTLGHALGETQRAQLVTWLKGNTTGAASIRAGLPTSWTAGDKTGSGGYGTTNDIAVIWPQGRAPLVLVTYFTQPQQNAESRRDVLASAARIIAEGL

SEQ ID NO: 9 - protein, 532 aa, Artificial Sequence
  fusion CTXM16-6G-TEM36
QTSAVQQKLAALEKSSGGRLGVALIDTADNTQVLYRGDERFPMCSTSKVMAAAAVLKQSETQKQLLNQPVEIKPADLVNYNPIAEKHVNGTMTLAELSAAALQYSDNTAMNKLIAQLGGPGGVTAFARAIGDETFRLDRTEPTLNTAIPGDPRDTTTPRAMAQTLRQLTLGHALGETQRAQLVTWLKGNTTGAASIRAGLPTSWTAGDKTGSGGYGTTNDIAVIWPQGRAPLVLVTYFTQPQQNAESRRDVLASAARIIAEGLGGGGGGHPETLVKVKDAEDQLGARVGYIELDLNSGKILESFRPEERFPMVSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERDRQIAEIGASLIKHW

SEQ ID NO: 10 - protein, 537 aa, Artificial Sequence
  fusion TEM36 - G(EAAAK)2 - CTXM16
HPETLVKVKDAEDQLGARVGYIELDLNSGKILESFRPEERFPMVSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERDRQIAEIGASLIKHWGEAAAKEAAAKQTSAVQQKLAALEKSSGGRLGVALIDTADNTQVLYRGDERFPMCSTSKVMAAAAVLKQSETQKQLLNQPVEIKPADLVNYNPIAEKHVNGTMTLAELSAAALQYSDNTAMNKLIAQLGGPGGVTAFARAIGDETFRLDRTEPTLNTAIPGDPRDTTTPRAMAQTLRQLTLGHALGETQRAQLVTWLKGNTTGAASIRAGLPTSWTAGDKTGSGGYGTTNDIAVIWPQGRAPLVLVTYFTQPQQNAESRRDVLASAARIIAEGL

SEQ ID NO: 11 - DNA, 4158 bp, Artificial Sequence
  Vector and clone sequence TEM36
acattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgtcggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaattttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacacagaattccgaaacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagctcaccctgagacacttgtcaaggttaaggatgccgaagatcaactaggagcaagggttggttacattgaactggacttgaatagtggtaagattttagagagcttcagacctgaggagcgattcccaatggttagcacctttaaggtactcctatgcggagcagtgttgtcaagagtcgatgctggtcaagagcagttaggaaggagaatccattattcacagaacgacttggttgaatattccccagtgactgagaaacacttgacagatggaatgacagttcgtgagttgtgttctgctgctatcactatgtctgataatacagctgcaaatctgctgctgacgacaatcggtggtcccaaagagttgaccgcttttctgcataacatgggcgatcacgtaacaagacttgaccgttgggagcctgaacttaacgaagccatccccaatgacgaaagagatactactatgccagctgcaatggctaccactctgcgtaagttgcttactggtgaattgttgacgttagcttctaggcaacaattaatcgactggatggaagctgataaagttgctggcccattgttgagaagtgctttgccggcaggatggttcattgcagacaaatcgggagccggtgaaagaggttcaagaggtattatagctgcattgggtcctgacggtaaaccatccagaattgtggtcatctacactactggttcccaggctacaatggacgagcgtgatagacaaattgccgaaataggagcatccctaatcaaacattggtgaggggcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagagcaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctcttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggacc

SEQ ID NO: 12 - DNA, 900 bp, Escherichia coli
actatcgcttccattgctgctaaggaagagggtgtctctctcgagaaaagagaggccgaagctcagacgtcagccgtgcagcaaaagctagctgcgctagaaaagtcgagcggcggtcggcttggtgttgcattgattgacaccgccgacaacacccaggtattgtatcgtggggatgaacgcttcccgatgtgctctacatctaaagttatggcggccgctgcagtgctgaaacaatctgaaacgcagaagcaattgctgaatcagcccgttgagattaagccggctgatctggtgaattacaatccgattgccgaaaaacacgtgaacggtaccatgacgctggctgaactgagtgctgcagcgcttcaatatagcgataacacggccatgaataagctgatcgcccagttaggcggtccgggaggagtgaccgccttcgcgcgtgccataggtgatgaaactttccgtctggatcgcacggaaccgacactgaacacagcgatcccgggtgatccacgtgataccaccacgccgcgcgcaatggcacagaccctgcgccaactgacgctcggccatgcgctgggggagacacaacgtgcgcagctggttacttggctgaaagggaatacaactggggctgcgtcgattcgtgctggactgccgacaagttggactgccggcgataaaactgggtcaggtggatatggcactaccaatgatatcgcagtgatatggccgcagggtagagcgcctcttgttttggttacctacttcacgcaaccccagcagaacgcggagtcacgtcgtgacgtgcttgcatcagccgctcggatcatagctgaaggtttgtaaaagcggccgccgctcaagaggatgtcagaatgccatttgcctgag

SEQ ID NO: 13 - DNA, 1680 bp, Artificial Sequence
  fusion 1
actatcgcttccattgctgctaaggaagagggtgtctctctcgagaaaagagaggccgaagctcaccctgagacacttgtcaaggttaaggatgccgaagatcaactaggagcaagggttggttacattgaactggacttgaatagtggtaagattttagagagcttcagacctgaggagcgattcccaatggttagcacctttaaggtactcctatgcggagcagtgttgtcaagagtcgatgctggtcaagagcagttaggaaggagaatccattattcacagaacgacttggttgaatattccccagtgactgagaaacacttgacagatggaatgacagttcgtgagttgtgttctgctgctatcactatgtctgataatacagctgcaaatctgctgctgacgacaatcggtggtcccaaagagttgaccgcttttctgcataacatgggcgatcacgtaacaagacttgaccgttgggagcctgaacttaacgaagccatccccaatgacgaaagagatactactatgccagctgcaatggctaccactctgcgtaagttgcttactggtgaattgttgacgttagcttctaggcaacaattaatcgactggatggaagctgataaagttgctggcccattgttgagaagtgctttgccggcaggatggttcattgcagacaaatcgggagccggtgaaagaggttcaagaggtattatagctgcattgggtcctgacggtaaaccatccagaattgtggtcatctacactactggttcccaggctacaatggacgagcgtgatagacaaattgccgaaataggagcatccctaatcaaacattggggaggaggtggaggtggacagacgtcagccgtgcagcaaaagctagctgcgctagaaaagtcgagcggcggtcggcttggtgttgcattgattgacaccgccgacaacacccaggtattgtatcgtggggatgaacgcttcccgatgtgctctacatctaaagttatggcggccgctgcagtgctgaaacaatctgaaacgcagaagcaattgctgaatcagcccgttgagattaagccggctgatctggtgaattacaatccgattgccgaaaaacacgtgaacggtaccatgacgctggctgaactgagtgctgcagcgcttcaatatagcgataacacggccatgaataagctgatcgcccagttaggcggtccgggaggagtgaccgccttcgcgcgtgccataggtgatgaaactttccgtctggatcgcacggaaccgacactgaacacagcgatcccgggtgatccacgtgataccaccacgccgcgcgcaatggcacagaccctgcgccaactgacgctcggccatgcgctgggggagacacaacgtgcgcagctggttacttggctgaaagggaatacaactggggctgcgtcgattcgtgctggactgccgacaagttggactgccggcgataaaactgggtcaggtggatatggcactaccaatgatatcgcagtgatatggccgcagggtagagcgcctcttgttttggttacctacttcacgcaaccccagcagaacgcggagtcacgtcgtgacgtgcttgcatcagccgctcggatcatagctgaaggtttgtaaaagcggccgctcaagaga

SEQ ID NO: 14 - DNA, 1634 bp, Artificial Sequence
  fusion 2
ctcgagaaaagagaggccgaagctcagacgtcagccgtgcagcaaaagctagctgcgctagaaaagtcgagcggcggtcggcttggtgttgcattgattgacaccgccgacaacacccaggtattgtatcgtggggatgaacgcttcccgatgtgctctacatctaaagttatggcggccgctgcagtgctgaaacaatctgaaacgcagaagcaattgctgaatcagcccgttgagattaagccggctgatctggtgaattacaatccgattgccgaaaaacacgtgaacggtaccatgacgctggctgaactgagtgctgcagcgcttcaatatagcgataacacggccatgaataagctgatcgcccagttaggcggtccgggaggagtgaccgccttcgcgcgtgccataggtgatgaaactttccgtctggatcgcacggaaccgacactgaacacagcgatcccgggtgatccacgtgataccaccacgccgcgcgcaatggcacagaccctgcgccaactgacgctcggccatgcgctgggggagacacaacgtgcgcagctggttacttggctgaaagggaatacaactggggctgcgtcgattcgtgctggactgccgacaagttggactgccggcgataaaactgggtcaggtggatatggcactaccaatgatatcgcagtgatatggccgcagggtagagcgcctcttgttttggttacctacttcacgcaaccccagcagaacgcggagtcacgtcgtgacgtgcttgcatcagccgctcggatcatagctgaaggtttgggaggaggaggaggaggacaccctgagacacttgtcaaggttaaggatgccgaagatcaactaggagcaagggttggttacattgaactggacttgaatagtggtaagattttagagagcttcagacctgaggagcgattcccaatggttagcacctttaaggtactcctatgcggagcagtgttgtcaagagtcgatgctggtcaagagcagttaggaaggagaatccattattcacagaacgacttggttgaatattccccagtgactgagaaacacttgacagatggaatgacagttcgtgagttgtgttctgctgctatcactatgtctgataatacagctgcaaatctgctgctgacgacaatcggtggtcccaaagagttgaccgcttttctgcataacatgggcgatcacgtaacaagacttgaccgttgggagcctgaacttaacgaagccatccccaatgacgaaagagatactactatgccagctgcaatggctaccactctgcgtaagttgcttactggtgaattgttgacgttagcttctaggcaacaattaatcgactggatggaagctgataaagttgctggcccattgttgagaagtgctttgccggcaggatggttcattgcagacaaatcgggagccggtgaaagaggttcaagaggtattatagctgcattgggtcctgacggtaaaccatccagaattgtggtcatctacactactggttcccaggctacaatggacgagcgtgatagacaaattgccgaaataggagcatccctaatcaaacattggtgaggggcggccgc

SEQ ID NO: 15 - DNA, 1648 bp, Artificial Sequence
  fusion 3
ctcgagaaaagagaggccgaagctcaccctgagacacttgtcaaggttaaggatgccgaagatcaactaggagcaagggttggttacattgaactggacttgaatagtggtaagattttagagagcttcagacctgaggagcgattcccaatggttagcacctttaaggtactcctatgcggagcagtgttgtcaagagtcgatgctggtcaagagcagttaggaaggagaatccattattcacagaacgacttggttgaatattccccagtgactgagaaacacttgacagatggaatgacagttcgtgagttgtgttctgctgctatcactatgtctgataatacagctgcaaatctgctgctgacgacaatcggtggtcccaaagagttgaccgcttttctgcataacatgggcgatcacgtaacaagacttgaccgttgggagcctgaacttaacgaagccatccccaatgacgaaagagatactactatgccagctgcaatggctaccactctgcgtaagttgcttactggtgaattgttgacgttagcttctaggcaacaattaatcgactggatggaagctgataaagttgctggcccattgttgagaagtgctttgccggcaggatggttcattgcagacaaatcgggagccggtgaaagaggttcaagaggtattatagctgcattgggtcctgacggtaaaccatccagaattgtggtcatctacactactggttcccaggctacaatggacgagcgtgatagacaaattgccgaaataggagcatccctaatcaaacattggggagaggcggccgctaaagaggcagcagcaaaacagacgtcagccgtgcagcaaaagctagctgcgctagaaaagtcgagcggcggtcggcttggtgttgcattgattgacaccgccgacaacacccaggtattgtatcgtggggatgaacgcttcccgatgtgctctacatctaaagttatggcggccgctgcagtgctgaaacaatctgaaacgcagaagcaattgctgaatcagcccgttgagattaagccggctgatctggtgaattacaatccgattgccgaaaaacacgtgaacggtaccatgacgctggctgaactgagtgctgcagcgcttcaatatagcgataacacggccatgaataagctgatcgcccagttaggcggtccgggaggagtgaccgccttcgcgcgtgccataggtgatgaaactttccgtctggatcgcacggaaccgacactgaacacagcgatcccgggtgatccacgtgataccaccacgccgcgcgcaatggcacagaccctgcgccaactgacgctcggccatgcgctgggggagacacaacgtgcgcagctggttacttggctgaaagggaatacaactggggctgcgtcgattcgtgctggactgccgacaagttggactgccggcgataaaactgggtcaggtggatatggcactaccaatgatatcgcagtgatatggccgcagggtagagcgcctcttgttttggttacctacttcacgcaaccccagcagaacgcggagtcacgtcgtgacgtgcttgcatcagccgctcggatcatagctgaaggtttgtaaaagcggccgc

(19) United States
(12) Patent Application Publication
(10) Pub. No.: US 2017/0354706 A1
(43) Pub. Date: Dec. 14, 2017

(54) HYBRID PROTEINACEOUS MOLECULE CAPABLE OF INHIBITING AT LEAST ONE ANTIBIOTIC AND PHARMACEUTICAL COMPOSITION CONTAINING IT

(71) Applicant: AZURRX SAS, Langlade (FR)

(72) Inventors: Daniel DUPRET, Calvisson (FR); Mathieu SCHUE, Raphele-les-arles (FR); Philippe JAIS, Issy-les-Moulineaux (FR)

(21) Appl. No.: 15/519,424

(22) PCT Filed: Oct. 13, 2015
(86) PCT No.: PCT/FR2015/052756

§ 371 Date: Apr. 14, 2017

(30) Foreign Application Priority Data
Oct. 16, 2014 (FR) 1459935

(51) Int. Cl. A61K 38/00; C12N 15/62; C12N 9/86; A61K 9/00; A61K 47/42

(52) U.S. Cl. CPC A61K 38/005; A61K 9/0002; A61K 47/42; C12N 9/86; C12N 15/62

(57) ABSTRACT

The invention relates to a hybrid proteinaceous molecule comprising at least two proteins capable of inhibiting the activity of at least one antibiotic, the proteins each having different biochemical properties and being bonded to one another. The hybrid proteinaceous molecule inhibits the activity of a least one antibiotic in order to reduce the intestinal side effects of antibiotics, such as severe diarrhoea caused by the antibiotics, and nosocomial infections secondary to parenteral antibiotic therapy.

### FIELD OF THE INVENTION

The invention concerns the prevention of the adverse effects of antibiotics, secondary to their action on the gastrointestinal flora and the selection of resistant bacteria strains.

### BACKGROUND

The liver may excrete a great many parenterally administered antibiotics into the bile in active, unmodified active form or as active metabolites. These antibiotics may be reabsorbed in the ileum and thereby return to the liver via the portal vein to be eventually excreted. This circulation is said to be enterohepatic.

When excreted in the digestive tract in unmodified active form or as active metabolites, these antibiotics may exert selective pressure on the bacteria populations forming the normal micro-flora of the intestine and colon. Such microbial selection has two main consequences: the occurrence of acute diarrhoea induced by the antibiotics and the selection and dissemination of resistant bacteria strains.

The onset of acute diarrhoea caused by antibiotics is commonly observed with parenteral broad-spectrum antibiotics. The frequency and severity are determined by various factors including the duration of the treatment, the activity spectrum of the antibiotic used and the dose (Aires, Kohler et al., 1999). Acute diarrhoea induced by antibiotics is thereby observed in about 5-10% of all patients receiving antibiotic therapy with amoxicillin, 10-25% with an amoxicillin-clavulanic acid association and 2-5% with 3^{rd} generation cephalosporins, fluoroquinolones, azithromycin, clarithromycin, erythromycin and tetracyclines (Gilbert, 1994; Bartlett, 1996, Hogenauer, Hammer et al., 1998; Wistrom, Norrby et al., 2001).

Although the majority of the cases of acute diarrhoea caused by antibiotics are benign, they may be accompanied by severe colitis in 10-20% of all cases. In most cases, this colitis is secondary to the selection of Clostridium difficile, a spore-forming, Gram-positive anaerobic bacteria resistant to most antibiotics (Gerding, 1989; Anand, Bashey et al., 1994). The pathogenicity of this bacteria is related to the production of both toxins A and B. These toxins induce an intense inflammatory reaction with the recruitment of neutrophils in the lamina propria and in the most severe forms, pseudo-membranous colitis, so named because of the appearance of the colorectal mucosa (Kelly, Pothoulakis et al., 1994). The mortality with pseudo-membranous colitis is high, especially in young children and the elderly. These colitises were responsible for over 7200 deaths in the United-States in 2010 (Sherry, Murphy et al., 2012). Other pathogens may be responsible for acute diarrhoea of varying severity induced by antibiotics, such as Klebsiella oxytoca, Clostridium perfringens type A, Straphylococcus aureus and Candida albicans (Sparks, Carman et al., 2001; Gorkiewicz, 2009).

The selection pressure that antibiotics exert on the intestinal micro-flora also contributes to the spread of resistant bacteria in the environment. The majority of the infections caused by these microorganisms in hospitals, also known as nosocomial infections, are particularly common and severe in intensive care and surgical units. In particular, they involve catheter infections, urinary tract infections, lung diseases and post-operative infections. They are mainly caused by Gram-negative enterobacteria, coagulase-positive or negative staphylococci, enterococci and Candida albicans (Richards, Edwards et al., 2000).

Nosocomial infections are an important cause of death and a major health issue. The emergence of resistant bacteria, secondary to the widespread use of antibiotic treatment, complicates the treatment of these nosocomial infections and represents a considerable cost to society. In Europe, nosocomial infections are directly responsible for approximately 25,000 deaths each year and an additional cost in healthcare and productivity losses estimated at over 1.5 billion euros per year (ECDC/EMEA Joint Working Group, 2009). In the United States, about 1.7 million nosocomial infections are directly or indirectly responsible for almost 99,000 deaths each year (Klevens, Edwards et al., 2007).

Antibiotics, in particular tetracylines, are extensively used in veterinary medicine in the treatment of livestock and pets. In addition, the use of these antibiotics is allowed in the United States to favour the growth and weight gain in livestock. Of course, this intensive use participates in the dissemination of resistant bacteria in the environment (Institute of Medicine, 1988; Committee on Drug Use in Food Animals, 1999, Georgetown University Center for Food and Nutrition Policy, 1999).

Beta-Lactams

Beta-lactams inhibit the transpeptidases and transglycosylases involved in the formation of peptidoglycane, an essential component of the cell wall of Gram-positive and Gram-negative bacteria (Walsh, 2000). They have a beta-lactam ring, as well as variable lateral chains (for example, 1^{st}, 2^{nd}, 3^{rd} and 4^{th} generation cephalosporins) that modify their pharmacokinetic properties and the efficacy of their antimicrobial spectrum (Turner, 2005). This class includes the penicillins, cephalosporins, carbapenems and monobactams).

Beta-lactam antibiotics are the most commonly prescribed antimicrobials. They are extensively used clinically for different types of infection (for example, ear, respiratory and gastrointestinal infections). Several routes of administration may be used but the parenteral route is preferred for severe sepsis, usually treated in hospitals. They are frequently associated with other classes of antibiotics for serious infections, such as the association of amoxicillin/clavulanic acid with a macrolide in cases of atypical pneumonia.

The intensive use of beta-lactam antibiotics has favoured the emergence of bacteria resistance. The main mechanism is the acquisition of beta-lactamases, enzymes able to hydrolyse their beta-lactam core. To date, there are several hundred or even thousands of different beta-lactamases that can be grouped into families according to the sequence homology (substrate and inhibitors). These enzymes are generally classified according to their preferential substrate (penicillinase, cephalosporinase, imipenemase, etc.). In addition, beta-lactamases have evolved to become resistant to inhibitors or broaden their action spectrum on the latest beta-lactams (Turner, 2005; Drawz and Bonomo, 2010). These inhibitors (clavulanic acid, sulbactam, etc.) are generally administered simultaneously with beta-lactams and irreversibly bond with bacterial beta-lactamases.

Ambler's classification illustrates the huge diversity of these beta-lactamase enzymes. They are grouped in four families (A, B, C and D) according to their nucleotide sequence (Ambler, 1980). Groups A, C and D have a catalytic serine while group B includes the zinc-dependent metallo-beta-lactamases.

Class A beta-lactamases include several sub-families, including TEM type enzymes (Bush and Jacoby, 1997), ancestral enzymes for resistance to penicillin and aminopenicillin (for example, ampicillin, amoxicillin, becampicillin). TEM-36 is an IR-TEM, that is, a clavulanic acid-resistant beta-lactamase (Zhou, Bordon et al., 1994; Henquell, Chanal et al., 1995; Chaibi, Sirot et al., 1999); TEM-36 or IRT-7 identifier on the Lahey Clinic site (http://www.lahey.org/Studies/temtable.asp). Clavulanic acid is clinically used in association with amoxicillin to broaden the antibiotic spectrum to germs that have acquired a classic beta-lactamase (TEM-1). Under the pressure of selection, the TEM have gradually become resistant to the inhibitor by mutating several amino acids that are currently well characterised (Chaibi, Sirot et al., 1999).

Among the class A beta-lactamases, we also find cefotaximases, enzymes that evolved from TEM to broaden their spectrum to 3^{rd} generation cephalosporins (Salverda, De Visser et al., 2010). For example, the enzyme CTX-M16 (Genbank identifier of the protein: AAK32961) preferentially hydrolyses cefotaxime when compared with different penicillins (Bonnet, Dutour et al., 2010).

The PC1 enzyme (UniProtKB/Swiss-Prot identifier of the protein: P00807) encoded by the gene blaZ is a class-A beta-lactamase catalytic serum (Ambler, 1975; Knott-Hunziker, Waley et al., 1979; Kemal and Knowles, 1981; Hertzberg and Moult, 1987). This enzyme produced by coagulase-positive Staphylococcus aureus and methi-R Bacillus subtilis hydrolyses the beta-lactam ring of methicillin (Novick, 1963).

Aminoglycosides

Aminoglycosides, also referred to aminosides, are amino sugars linked by a glycoside bridge to a central aminocyclitol core. The structure of this central core is used to distinguish three groups: streptomycins (streptomycin), desoxystreptamins (Kanamycin, Amikacin, Gentamicin) and fortimicins (Dactamicin).

The theoretical antibiotic spectrum of aminoglycosides is very wide and includes aerobic Gram-negative bacteria (bacilli, cocci and coccobacilli), positive or negative coagulase staphylococci and Gram-positive bacilli. In addition, streptomycin is active on Mycobacterium tuberculosis and amikacin on atypical mycobacteria (Mycobacterium avium intracellulare, etc.), as well as Nocardia asteroids. The aminoglycosides have a synergic activity with beta-lactams and vancomycin.

The aminoglycosides are usually administered parenterally, since little or none is absorbed in the digestive tract. They are mainly eliminated in unchanged form, mainly in the urine by glomerular filtration (85 to 90% of the dose administered is eliminated within 24 hours), and to a lesser extent in the bile without entero-hepatic circulation (about 0.5 to 2% of the dose administered according to the antibiotic) (Leroy, Humbert et al., 1978). This gastric excretion of aminoglycosides results in the selection of resistant bacteria, exposing to a slightly increased risk of pseudomembranous colitis by Clostridium difficile (Arnand, Bashey et al., 1994).

The bactericidal effect of aminoglycosides is fast on many pathogens. Aminoglycosides fix on the 30S ribosomal prokaryote sub-unit and alter the accuracy of the bacterial translation (Poehlsgaard and Douthwaite, 2005). Most of the aminoglycosides bond with 16S ribosomal RNA, the RNA component of the 303 ribosomal sub-unit, at the decoding site (site A).

Bacteria may develop resistance to aminoglycosides by three main mechanisms that may coexist in the same cell (Ramirez and Tolmasky, 2010). A first chromosomic mechanism leads to a reduction of the affinity of the aminoglycoside for its ribosomal target, 16S ribosomal RNA, through its methylation (Doi and Arakawa, 2007). A second mechanism is based on cell permeability failure by modification of the membrane permeability (Hancock, 1981) or an aminoglycoside efflux outside of the cell by an active mechanism (Aires, Kohler et al., 1999). Finally, the enzyme inactivation is the most frequently observed mechanism. Genes encoding these enzymes are carried by plasmids and/or transposons. The resistance is thereby transferable and is often epidemic in hospitals. These enzymes catalyse the irreversible bonding of aminoglycoside with different chemical groups and are grouped in three classes (Shaw, Rather et al., 1993; Ramirez and Tolmasky, 2010). The phosphotransferases (APH) which catalyse a transfer reaction of a phosphate radical of ATP or GTP, the nucleotideyltransferases (ANT) enable the transfer of an adenyl radical and use ATP as a substrate and the acetyltransferases (AAC) transfer an acetyl radical using acetyl-CoA as substrate. Each enzyme class has different sub-classes according to the substituent of the aminoglycoside. The same enzyme can inactivate several aminoglycosides with identical molecular sites.

AAC(6′)-lb-cr (Genbank identifier of the protein: ABC17627.1) is an enzyme from the N-acetyltransferase family naturally produced by enterobacteria. It catalyses the acetylation of an —NH₂ group in position 6′, giving this enzyme a resistance profile identical to other enzymes in the sub-class AAC(6′) (Robicsek, Strahilevitz et al., 2006). Like other enzymes in the family AAC(6′), AAC(6′)-lb-cr is active on a wide range of aminoglycosides including amikacin and the C1a and C2 enantiomers of gentamicin, but has a very low activity on the C1 enantiomer of gentamicin (Robicsek, Strahilevitz et al., 2006). AAC(6′)lb-cr, is probably due to the evolution of the enzyme AAC(6′)-lb by mutation of Trp102Arg and Asp179Tyr residues. This results in the acquisition of enzyme activity on ciprofloxacin, a fluoroquinolone, an activity that is considerably lower for norfloxacin and pefloxacin.

Fluoroquinolones

Fluoroquinolones (for example, norfloxacin, ofloxacin, ciprofloxacin and pefloxacin) form a large class of synthetic bactericidal antibiotics derived from quinolones by chemical modifications, in particular the addition of a fluorine atom.

The fluoroquinolones are broad spectrum antibiotics which differ from one antibiotic to another. The spectrum of fluoroquinologes includes Gram-negative bacilli (Salmonella spp., Escherichia spp., Shigella spp., Proteus spp., Enterobacter spp., Helicobacter pylori), Gram-positive cocci (positive and negative coagulase staphylococcus, streptococcus, enterococcus), Gram-negative cocci (gonococcus, meningococcus) and Gram-positive bacilli.

The tissue distribution of fluoroquinolones is excellent even in the cerebrospinal fluid, the prostrate, bones and bile. Fluoroquinolones are thereby widely used in case of tissue infection (meningitis, pneumonia, bone and joint infection, infection of the upper urinary tract or prostate infection, etc). However, the serum level of fluoroquinolones is often low and may even be lower than the MIC (Minimum Inhibitory Concentration) of certain germs, favouring the emergence of bacterial resistance.

The excretion of fluoroquinolones depends on the product used. It is mainly hepatic for pefloxacin, while it is mainly renal for oflaxacin and other fluoroquinolones. Their biliary and digestive elimination accounts for the risk of pseudomembranous colitis by Clostridium difficile, in particular by the strain Bl/NAP1/027, which is especially virulent (Vardakas, Konstantelias et al., 2012; Deshpande, Pasupuleti et al., 2013; Slimings and Riley, 2013).

Fluoroquinolones target DNA gyrase and bacterial topoisomerase II and IV. They form an irreversible complex between these enzymes and bacterial DNA, which prevents DNA replication and causes the death of the bacteria.

Four quinolone resistance mechanisms have been characterised (Robicsek, Jacoby et al., 2006): (i) an increased activity of efflux pumps, provoking a reduction in the intracellular concentration of the antibiotic (Morita, Kodama et al., 1998), (ii) the production of proteins that bind to DNA gyrase or topoisomerase IV, protecting them as well as the fixation of fluoroquinolones (Robicsek, Jacoby et al., 2006), (iii) a modification of the DNA gyrase or topoisomerase IV responsible for high level resistance by reduction of the affinity of the fluoroquinolones for their targets (Robicsek, Jacoby et al., 2006), (iv) finally, the production of an aminoglycoside N-acetyltransferase, AAC(6′)lb-cr (Genbank identifier of the protein: ABC17627.1) capable of catabolising the modification of ciprofloxacin and, to a lesser degree, norfloxacin and pefloxacin (Robicsek, Strahilevitz et al., 2006).

These mechanisms of resistance, whose frequency is quickly increasing, have been identified in many bacteria species, including coagulase-positive Staphylococcus aureus, beta-haemolytic streptococci and enterococci (Robicsek, Jacoby et al., 2006).

Macrolides

Macrolides are a homogenous family of natural and semi-synthetic antibiotics with a relatively limited number of representatives (for example, erythromycin, spiramycin, josamycin, clarithromycin, roxithromycin, azithromycin and telithromycin).

The activity spectrum of macrolides is relatively broad and includes aerobic Gram-positive cocci (streptococcus, pneumococcus, enterococcus and straphylococcus) and anaerobic Gram-negative cocci, Gram-negative cocci, certain Gram-negative bacilli such as Helicobacter pylori, and different bacteria with a strict intracellular replication such as Legionella pneumophilia, Mycoplasma pneumoniae and Mycoplasma urealyticum, Clamydiae pneumoniae and Clamydiae trachomatis. However, enterobacteria are intrinsically resistant to macrolides since their outer cell membrane prevents the passage of hydrophobic molecules such as macrolides.

Macrolides are widely distributed in the body, with the exception of cerebrospinal fluid, the brain and urine. Elimination is mainly biliary, after metabolism by the cytochrome P450. The administration of macrolides is associated with an increased risk of pseudomembranous colitis secondary to the selection of Clostridium difficile (Gilbert, 1994; Bartlett, 1996; Hogenauer, Hammer et al., 1998; Wistrom, Norrby et al., 2001).

Macrolides are inhibitors, which bond reversibly with the 50S sub-unit of prokaryotic ribosomes, at site P. They thereby prevent the transfer and dissociation of the peptidyl-tRNA complex (transfer RNA) from site P to site A, and thereby inhibit the peptide chain elongation (Tenson, Lovmar et al., 2003).

Three resistance mechanisms to macrolides are known (Roberts, Sutcliffe et al., 1999; Roberts, 2008): (i) a change in the target by methylation or mutation of the bacterial 23S ribosomal RNA, forming 50S ribosomal RNA. This resistance mechanism, which is most often encountered, is macrolides, lincosamides and streptogramines B (Weisblum, 1995), (ii) production by cell pumps leaking the antibiotic outside of the cell, resulting in a decrease in the intracellular concentration, (iii) inactivation by enzymes (esterase erythromycin a phosphotransferase erythromycin) modifying the macrolides so that their affinity for the ribosome is greatly reduced. This type of resistance is also transmitted by mobile genetic elements.

Erythromycin esterase enzymes inactivate macrolides by hydrolysis of the macrolactone core. Most of these enzymes belong to two sub-classes: ereA (Ounissi and Courvalin, 1985) and ereB (Arthur, Autissier et al., 1986). Enzyme ereB (UniProtKB/Swiss-Prot identifier: P05789.1) with a very broad spectrum (erythromycin, clarithromycin, roxithromycin and azithromycin), although telithromycin is resistant to hydrolysis (Morar, Pengelly et al., 2012).

Tetracyclines

Cyclines or tetracyclines are a family of bactericidal antibiotics derived from tetracycline (for example chlorotetracycline, doxycycline, minocycline). These molecules have the characteristic of having four fused rings, hence the name.

The activity spectrum of tetracylines extends to many aerobic and anaerobic Gram-positive and Gram-negative bacteria. Tetracyclines are also active on unconventional pathogens such as Mycoplasma spp., Chlamydia spp., Rickettsia trepoinemes and some protozoa (Babesia divergens, Babesia microti, Theileria parva). The mycobacteria and Enterobacteriaceae Proteus and Pseudomonas are naturally resistant.

Most of the tetracyclines are eliminated by glomerular filtration in active form except for chlorotetracycline. Tetracyclines are also eliminated in the bile with enterohepatic cycle. The use of tetracyclines is also associated with an increased risk of pseudomembranous colitis secondary to the selection of Clostridium difficile (Gilbert, 1994; Bartlett, 1996; Hogenauer, Hammer et al., 1998; Wistrom, Norry et al., 2001).

Antibiotics from the cycline family inhibit the fixation of aminoacyl-tRNA of site A of the ribosomal 305 sub-unit and thereby inhibit the translation (Chopra and Roberts, 2001). At least three mechanisms of resistance have been identified: (i) the expression of efflux proteins, inducing a reduction in the intracellular concentration of tetracyclines, (ii) an enzyme modification of the molecular target, preventing the bonding of tetracyclines, (iii) inactivation by TetX (GenBank identifier of the protein: AAA27471.1), an NADPH-dependent oxyreductase tetracycline (Speer, Bedzyk et al., 1991). This enzyme induces bacterial resistance with respect to all tetracyclines.

Lincosamides

Lincosamides (lincomycin and its semi-synthetic derivative clindamycin) are bacteriostatic antibiotics that, although the chemical structure differs from that of the macrolides, have a similar mode of action and are grouped with the spectogramines B in a single family called MLS (macrolides, lincosamides and streptogramines).

The action spectrum of lincosamides covers the majority of Gram-positive bacteria (for example, Bacillus cereus, Corynebacterium diphtheria, Enterococcus faecium, MSSA and MRSA, B Staphylococcus, non-groupable Staphylococcus, Streptococcus pneumonia, Streptococcus pyogenes) with a few exceptions such as Streptococcus faecalis, as well as a great many anaerobic bacteria with the noteworthy exception of Clostridium difficile (for example, actinomyces, bacteroides, fusobacterium, Propionibacterium acnes). Most of the Gram-negative bacteria are resistant except for a few rare exceptions (for example, Bordetella pertussis, Campylobacter, Chlamydia, Helicobacter and Legionella).

Lincosamides are mainly eliminated by the kidney and to a lesser extent in the bile with the enterohepatic cycle. Their elimination from the gastrointestinal tract and their antibacterial activity account for the high frequency of pseudomembranous colitis by Clostridium difficile selection.

Like the macrolides, the lincosamides inhibit bacterial translation by reversibly bonding with the 50S ribosome sub-unit, at sites A and P (Tu, Blaha et al., 2005). Three resistance mechanisms acquired with lincosamides are known (Roberts, Sutcliffe et al., 1999): (i) a change in the target by methylation or mutation of the bacterial 23S ribosomal RNA, comprising the 50S ribosomal RNA. This resistance mechanism, the one most frequently encountered, is common to macrolides, lincosamides and B streptogramines (Weisblum, 1995), (ii) expression by the bacteria of pumps that efflux the antibiotic outside of the cell, provoking a reduction in its intracellular concentration, (iii) enzyme inactivation of lincosamides reducing their affinity for their molecular target.

Enzymes able to inactivate lincosamides belong to the family of lincomycin nucleotidyltransferases Inu(A) or lin (A) in staphylococci, Inu(B) or lin(B) in Enterococcus faecium, and linB-like in anaerobics (Leclercq, Brisson-Noel et al., 1987; Bozdogan, Berrezouga et al., 1999). For example, the enzyme Inu(B) (UniProtKB/TrEMBLication of the protein: Q9WVY4) catalyses the transfer of an adenyl residue on the hydroxyl group in position 3 of the lincomycin and clindamycin (Bozdogan, Berrezouga et al., 1999).

Counter the Side Effects of These Antibiotics

The prior art is familiar with inhibitors of antibiotics that aim at reducing the adverse reactions previously reported in the intestine. These inhibitors are essentially targeted against beta-lactams and primarily consist of beta-lactamases administered orally for their dissemination in the intestinal tract.

Patent EP0671942 relates to a medical application, a medical procedure and a pharmaceutical preparation. This invention can target the action of beta-lactams administered parenterally and reduce the adverse reactions due to the inactivation of a portion of the antibiotic in the digestive tract, by administering an enzyme such as beta-lactamase orally, either separately of simultaneously with the antibiotic. This leads to the breakdown of the antibiotic.

The EP2086570 application uses, with a similar method, a class A beta-lactamase, more specifically the P1A enzyme, to reduce the intestinal side effects associated with antibiotic therapy combining a beta-lactam and a beta-lactamase inhibitor.

The efficacy of P1A beta-lactamase was evaluated pre-clinically and clinically. This enzyme hydrolyses the aminopenicillins (for example, amoxicillin) and the ureidopenicillins (for example, piperacillin), but is sensitive to beta-lactam inhibitors. Oral administration in the dog, simultaneously with a parenteral administration of ampicillin reduced, in a dose-dependent manner, the amount of ampicillin detected in the jejunal lumen in these animals. In addition, the presence of P1A beta-lactamase was not detected in the circulation and the serum concentrations of ampicillin were not significantly modified (Harmoinen, Vaali et al., 2003; Harmoinen, Mentula et al., 2004). The oral administration of this enzyme in the mouse receiving a parenteral administration of piperacillin very significantly reduced the number of resistant microorganisms to this antibiotic in the faeces (VRE-Enterococcus faecium, Klebsiella pneumoniae and Candida glabrata) (Stiefel, Pultz et al., 2003; Mentula, Harmoinen et al., 2004).

Finally, a clinical trial demonstrated that the oral administration of P1A beta-lactamase reduced the number of isolates of Enterobacteriaceae resistant to ampicillin in healthy volunteers after the intravenous administration of ampicillin, as well as the number of Enteriobacteriaceae isolates resistant to other class of antibiotics, in particular resistant to the tetracyclines (Tarkkanen, Heinonen et al., 2009).

Recombinant beta-lactamase enzymes belonging to other classes of beta-lactamases have also been developed. Patent EP2038411 describes a metallo-beta-lactamase mutant and its synthesis to reduce the intestinal side effects in patients receiving carbapenemes.

The prior art also includes dosage forms to target the delivery of these enzymes in the intestine. Patent FR2843303 describes multi-particle dosage forms for oral administration for delivery limited to the colon of enzymes able to inhibit macrolides such as erythromycin esterase.

### Disadvantage of the Solutions in the Prior Art

The solutions in the prior art use enzymes only targeting one type of antibiotic, beta-lactamases breaking down beta-lactams or erythromycin esterase inhibiting macrolides.

Different antibiotics are generally used on hospital patients and are even combined in the same patient. The inhibition of a single class of antibiotics only has a modest or minimum effect on the emergence of resistant bacterial strains.

By way of example, severe infections with Gram-negative bacilli are frequently treated with an association of 3^{rd} generation cephalosporin and an aminoglycoside, whereas atypical pneumonia is readily treated with an association of amoxicillin-clavulanic acid and a macrolide or tetracycline.

Since the antibiotic inhibitors described in the prior art are directed against a single class of antibiotics, they are not effective in preventing the emergence of resistant bacteria strains in an environment where different classes of antibiotics are simultaneously used, in particular in hospitals.

### Solution Provided by the Invention

The present invention proposes, in its most widely accepted sense, to overcome the drawbacks of the prior art by providing a hybrid protein molecule comprising at least two proteins capable of inhibiting the activity of at least one antibiotic, each protein having different biochemical properties, said proteins being related. In one embodiment, said proteins capable of inhibiting the activity of at least one antibiotic are linked together covalently.

The hybrid protein molecule, according to the invention, inhibits the activity of at least one antibiotic to reduce the intestinal side effects of antibiotics, such as acute diarrhoea caused by antibiotics and nosocomial infections secondary to the administration of parenteral antibiotics.

It is understood that the present invention has a broader spectrum of action compared with the solutions in the prior art.

Preferably, the hybrid protein molecule in the present invention is administered orally. The hybrid protein molecule according to the invention may comprise proteins capable of inhibiting many classes of antibiotics and, in particular, the associations most commonly used in clinical practice. Thus, the administration of the single hybrid protein molecule can target several antibiotics, thereby reducing the number of products prescribed.

Hybrid protein molecule refers to a hybrid protein formed by the artificial combination of two or more polypeptide chains.

Inhibition of the antibiotic activity refers to all processes leading to a reduction or suppression of the biological activity of the antibiotic considered. This includes, without limitation, the bonding of the antibiotic on another molecule in a specific manner (for example, with a monoclonal antibody or a fragment of a monoclonal antibody) or a non-specific manner (for example, by adsorption), a modification of the antibiotic by enzymatic or non-enzymatic addition of a chemical group or hydrolysis of the antibiotic by an antibiotic or non-antibiotic mechanism.

Advantageously, at least one of the proteins is an enzyme able to inhibit the activity of at least one antibiotic.

In another embodiment, each protein is an enzyme able to preferentially inhibit the activity of at least one antibiotic.

Advantageously, each protein preferentially inhibits the activity of different antibiotics.

In another embodiment of the present invention, the hybrid protein molecule comprises at least two proteins able to inhibit the activity of at least one antibiotic, each enzyme having different biochemical properties, said enzymes being bound together covalently.

It is understood that each enzyme preferentially breaks down two different antibiotics.

The present invention unexpectedly demonstrates that the hybrid protein molecules as defined above combine the respective functional activities of the proteins forming them, resulting in the expansion of their action spectrum and the potentiation of their efficacy in a hospital environment subject to high selection pressure.

Several types of proteins that inactivate one or more antibiotics may be used to form the hybrid protein molecule, including fragments of monoclonal antibodies (for example, monovalent or bivalent ScFv), hydrolase enzymes or enzymes catalysing other types of modifications.

Advantageously, the hybrid protein molecule according to the invention consists of one or several proteins able to inhibit the activity of an antibiotic, preferably an antibiotic selected from among a beta-lactam, an aminoglycoside, a fluoroquinolone, a macrolide, a tetracycline and/or a lincosamide. Preferably, each protein in the hybrid protein molecule inhibits the activity of different antibiotics. In one embodiment, the hybrid protein molecule comprises two proteins capable of inhibiting the activity of antibiotics belonging to the same class.

Advantageously, the sequence of at least one of the component proteins in the hybrid protein has a protein sequence homology of at least 40% with SEQ lD1 to SEQ lD7. This sequence homology is determined using CLUSTALW2 or CLUSTALOMEGA software with standard calculation parameters (Thompson, Higgins et al., 1994; Larkin, Blackshields et al., 2007). Preferably, this sequence homology is at last 50%, and even more preferably at least 60% with SEQ lD1 to SEQ lD7.

The component proteins or enzymes of the hybrid protein molecule may comprise zero, one or several glycosylations.

In one embodiment, the proteins or enzymes are combined into a single monocatenary protein.

In a preferred embodiment, the hybrid protein molecule comprises two enzymes inhibiting the activity of at least one antibiotic, one of the said enzymes is a beta-lactamase and one of the said enzymes is an enzyme chosen from among the beta-lactamases, the enzymes inhibiting an aminoglycoside, the enzymes inhibiting a fluoroquinolone, the enzymes inhibiting a macrolide, the enzymes inhibiting a tetracycline or the enzymes inhibiting a lincosamide, said enzymes being bonded together.

It is understood that the hybrid protein molecule may comprise:
two beta-lactamase enzymes bound together; or
an enzyme selected from among the beta-lactamases and an enzyme inhibiting an aminoglycoside, interconnected, said enzyme inhibiting an aminoglycoside being a phosphotransferase, a nucleotidyltransferase or an acetyltransferase; or
an enzyme selected from the beta-lactamases and an enzyme inhibiting a fluoroquinolone, interconnected, said enzyme inhibiting a fluoroquinolone being an aminoglycoside N-acetyltransferase; or
an enzyme selected from among the beta-lactamases and an enzyme inhibiting a macrolide, interconnected, said enzyme inhibiting a macrolide being an erythromycin esterase or an erythromycin phosphotransferase; or
an enzyme selected from among the beta-lactamases and an enzyme inhibiting a tetracycline, interconnected, said enzyme inhibiting a tetracycline being a NADPH-dependent oxydoreductase tetracycline; or
an enzyme selected from among the beta-lactamases and an enzyme inhibiting a lincosamide, interconnected, said enzyme inhibiting a lincosamide being a nucleotidyltransferase lincomycine.

The inventors have unexpectedly and surprisingly found that the efficacy of the hybrid protein molecule against its target antibiotics is equal to the enzymes comprising it taken alone.

These hybrid proteins may also be obtained artificially as a single protein chain resulting from the translation of a single reading frame obtained either by direct fusion of the respective reading frames of the protein components, or by means of a nucleotide sequence encoding an adapter consisting of one or several amino acids. This adapter, known to be inert and without specific biological action, may be flexible, semi-rigid or rigid (Chen, Zaro et al., 2013).

According to another embodiment, the proteins or enzymes are linked together covalently or by cross-linking.

These hybrid proteins may also be obtained artificially by cross-linking using chemical agents (Chen, Nielsen et al., 2013), enzymatic catalysis (Paguirigan and Beebe, 2007), exposure to ultraviolet light (Fancy and Kodadek, 1999), or any other method resulting in the formation of a covalent bond. Alternatively, such hybrid proteins may be obtained by assembly of one or several protein sub-units non-covalently by means of high-affinity ligands, such as for example the complex (strept)avidin/biotine (Schultz, Lin et al., 2000; Lin, Pagel et al., 2006; Pagel, Lin et al., 2006).

According to another embodiment, the present invention provides a pharmaceutical composition for human or veterinary use comprising the hybrid protein molecule according to the invention for use in the prevention of changes in the intestinal flora.

Advantageously, the invention provides a pharmaceutical composition for use in the prevention of nosocomial infections.

Advantageously, the invention provides a pharmaceutical composition for use in the prevention of diarrhoea associated with the administration of antibiotics.

According to a preferred embodiment, the pharmaceutical composition comprising the hybrid protein molecule according to the invention is a dosage form for oral administration.

It is understood that the invention provides a pharmaceutical composition comprising a hybrid protein molecule for the prevention of changes in the normal intestinal flora in order to prevent the spread of resistant bacteria in the environment, reduce the risk and/or severity of the nosocomial infections caused by multi-resistant microorganisms, and to reduce the frequency and severity of diarrhoea caused by antibiotics.

Advantageously, the pharmaceutical composition is for administration in humans, including paediatrics. It is understood that the pharmaceutical composition according to the present invention may be administered, preferably orally, before, at the same time or after the administration of one or several antibiotics.

In another embodiment, the pharmaceutical composition is for veterinary use in the prevention of the spread of bacterial resistance to antibiotics by pets and in farm animals receiving antibiotics.

The pharmaceutical composition according to the invention also includes any pharmaceutically acceptable substance such as adjuvants, excipients, stabilisers, salts, additives, binders, lubricants, coating agents, colorants, flavours, or any other conventionally used agent known to the person skilled in the art.

The pharmaceutical composition according to the invention may be in solid or liquid form, not limited in the form of a gel, syrup, capsule, tablet, suspension or emulsion.

According to one variant, the pharmaceutical composition comprises at least one gastro-protective agent.

Gastro-protective agent refers to an agent resistant to gastric juices for a delayed release of the hybrid protein molecule in the intestine, preferably in the duodenum and jejunum. The gastro-protective agent may also be a coating agent such as but not limited to the despolymethacrylates (for example, Eudragit®), cellulose-based polymers (cellulose ethers, for example Duodcell®, cellulose esters), polyvinyl acetate copolymers (for example Opandry®) or any other agent for coating or encapsulation or process known to the person skilled in the art.

According to another aspect, the invention includes a method for the production of the hybrid protein molecule in a living non-human recombinant organism.

A living recombinant organism refers to any cell capable of being genetically engineered to produce the hybrid protein molecule.

Without limitation, the term cell capable of being genetically modified refers to any prokaryotic cell (for example Escherichia coli), yeast (for example Saccharomyces cerevisiae, Pichia pastoris, Kluyveromyces lactis, Yarrowia lipolytica), insect cells infected or not infected with a baculovirus, mammalian cells (for example CHO, CHO-K1, HEK293, HEK293T).

### DESCRIPTION

The present invention will be better understood in the light of the description of the following non-limiting examples.

### DESCRIPTION OF THE FIGURES

FIG. 1 presents a protein sequence alignment of different beta-lactamases belonging to the family of IR-TEM (Amber Class A). The alignment was performed with CLUSTALW2 software on the https://www.ebi.ac.uk/Tools/msa/clustalw2 site using the default settings. FIG. 1 shows, by the alignment of several sequences, that the IR-TEM enzymes show >90% protein identity. These enzymes are relatively well preserved and share between 80 and 99% protein sequence identity and are also well characterised (Bonnet, 2004).

FIG. 2 presents a protein sequence alignment of different beta-lactamases belonging to the family of CTX-M cefotaximases (Amber Class A). The alignment was performed on the https://www.ebi.ac.uk/Tools/msa/clustalw2 site using the default settings on the software.

FIG. 3 provides an illustration of the PCR technique used to generate the hybrid proteins. Two proteins are first amplified separately with a common sequence (at the 3′ and 5′ ends respectively) constituting a linker. The two fragments are then hybridised to the level of the complementary DNA sequence (that of the linker) and the whole is re-amplified using external primers.

FIG. 4 describes all of the constructs used to express the TEM-36 (SEQ lD1), CTXM-16 (SEQ lD2), TEM36-GGGGGG-CTXM16 (SEQ lD8), CTXM16-GGGGGG-TEM36 (SEQ lD9) and TEM36-G(EAAAK)2-CTXM16 (SEQ lD 10) proteins in Pichia pastoris. Cloning into the vector pJexpress915 was performed in Xhol/Notl using, when necessary, moderate digestion to preserve the internal Notl sites at the coding DNA sequence of CTXM16.

FIG. 5 presents the DNA fragments cloned into Xhol/Notl coding for the TEM-36 (SEQ lD1), CTXM-16 (SEQ lD2), TEM36-GGGGGG-CTXM16 (SEQ lD8), CTXM16-GGGGGG-TEM36 (SEQ lD9) and TEM36-G(EAAAK)2-CTXM16 (SEQ lD 10) proteins and that were obtained by PCR using the pJexpress915-SEQ-F and pJexpress915-SEQ-R primers and the following programme (95° C. 30 sec-25 cycles [95° C. 30 sec-53° C. 45 sec-72° C. 1 min]-72° C. 5 min-4° C.).

FIG. 6 presents the hybrid proteins and their constituent enzymes TEM-36 (SEQ lD1) and CTMX-16 (SEQ lD 2) on a 12% SDS-PAGE gel as obtained by expression and purification in Pichia pastoris and after having undergone de-glycosylation treatment by EndoHf.

FIG. 7 describes all of the constructs used to express the TEM-36 (SEQ lD1), CTXM-16 (SEQ lD2) proteins and the hybrid proteins TEM36-GGGGG-CTXM16, CTXM-16-GGGGG-TEM36 in Escherichia coli. The cloning into the pET-26b(+) vector was carried out by Ndel/Hindlll.

FIG. 8 presents DNA fragments cloned into Ndel/Hindlll in the pET-26(+) encoding TEM-36 (SEQ lD1), CTXM-16 (SEQ lD2), TEM36-GGGGG-CTXM16, CTXM16-GGGGG-TEM36 and which were obtained by PCR using the T7-F and T7-R primers and the following programme (95° C. 30 sec-25 cycles [95° C. 30 sec-55° C. 45 sec-72° C. 1 min]-72° C. 5 min-4° C.).

FIG. 9 presents hybrid proteins (TEM36-G₅-CTXM16 and CTXM16-G₅-TEM36) and their constituent enzymes TEM-36 (SEQ lD1) and CTXM-16 (SEQ lD 2) on a 12% SDS-PAGE gel as obtained by expression in E. coli and purification from the periplasmic fraction.

FIG. 10 presents the DNA fragments encoding the AAC-6′-lb-cr (SEQ lD4), CTXM-16 (SEQ lD2) and AAC-H6-CTXM16 obtained in the construction of the fusion AAC-H6-CTXM16 (SEQ lD 18).

FIG. 11 presents the hybrid protein AAC-H6-CTXM16 and its constituent enzymes AAC-6′-lb-cr (SEQ lD4) and CTXM-16 (SEQ lD 2) on a 12% SDS-PAGE gel as obtained after expression and purification in E. coli for AAC-6′-lb-cr and the hybrid protein and P. pastoris for CTXM-16.

FIG. 12 presents DNA fragments encoding for EreB (SEQ lD5), TEM36 (SEQ lD1) and EreB-H6-TEM36, which were obtained in the construction of the fusion EreB-H6-CTXM16 (SEQ lD 20).

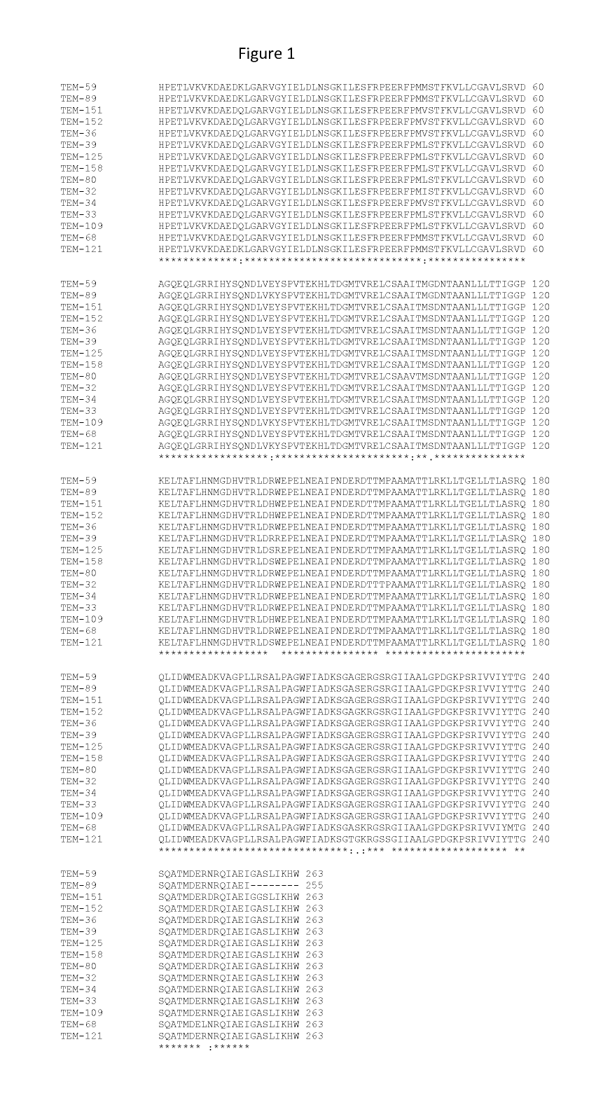

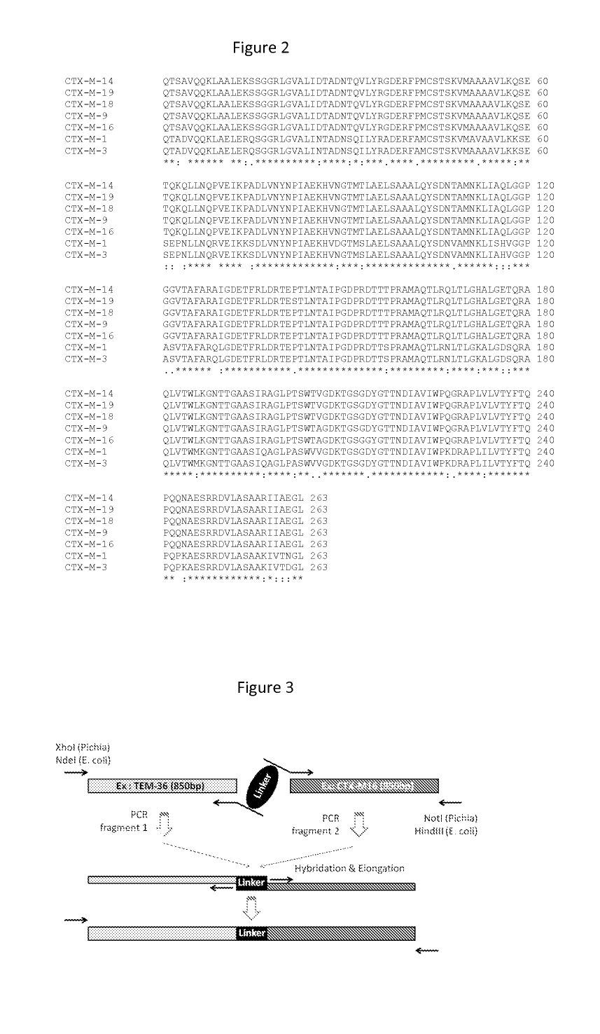

### DESCRIPTION OF THE LISTING OF SEQUENCES

Table 1 below summarises the listing of sequences and maps each SEQ-lD (ID No. in the table), the type of sequence, its name, its origin, its expression organism and its size.

TABLE 1
Identification of sequences 1 to 22 of the sequence listing

| | | | Identifier | | | | | |
|---|---|---|---|---|---|---|---|---|
| | ID | Common | (GeneBank/Swiss- | | Expression | | | |
| Type | No. | name | Prot/UnitProtKB) | Original host | microorganism | Length | | Description |
| Protein | 1 | TEM36 | Not available | Escherichia coli | E. coli./ | 263 | aa | β-lactamase |
| | | | | | P. pastoris | | | (aminopenicillinase) |
| Protein | 2 | CTXM6 | AAK32961 | Escherichia coli | E. coli./ | 263 | aa | β-lactamase |
| | | | | | P. pastoris | | | (cefotaximase) |
| Protein | 3 | PC1 | P00807 | Staphylococcus | E. coli./ | 257 | aa | β-lactamase |
| | | | | aureus | P. pastoris | | | (methicilli) |
| Protein | 4 | AAC-6′-lb-cr | ABC17627.1 | Escherichia coli | E. coli./ | 199 | aa | fluroquinolone acetylating |
| | | | | | P. pastoris | | | aminoglycoside |
| | | | | | | | | acetyltransferase |
| Protein | 5 | Ereb | P05789.1 | Escherichia coli | E. coli./ | 419 | aa | Erythromycine esterase |
| | | | | | P. pastoris | | | |
| Protein | 6 | TetX | AAA27471.1 | Bacteroides | E. coli./ | 388 | aa | Tetracycline oxydo-reductase |
| | | | | fragilis | P. pastoris | | | NADPH-dependent |
| Protein | 7 | LnuB (or | Q9WVY4 | Enterococcus | E. coli./ | 267 | aa | Lincomycine |
| | | linB) | | faecium | P. pastoris | | | nucleotidylttransferase |
| Protein | 8 | TEM36-G₆- | Not available | Artificial | Pichia | 532 | aa | Fusion TEM36 and CTXM16 |
| | | CTXM16 | | | pastoris | | | with flexible 6 Glycine liner |
| Protein | 9 | CTXM16- | Not available | Artificial | Pichia | 532 | aa | Fusion CTXM16 and TEM36 |
| | | G₆-TEM36 | | | pastoris | | | with flexible 6 Glycine linker |
| Protein | 10 | TEM36- | Not available | Artificial | Pichia | 537 | aa | Fusion TEM36 and |
| | | G(EAAAK)₂- | | | pastoris | | | CTXM16 with rigid linker |
| | | CTXM16 | | | | | | G(EAAAK)₂ |
| Nucleotide | 11 | TEM36 | Not available | Escherichia coli | E. coli./ | 4158 | bp | Total sequence of vector |
| | | | | | P. pastoris | | | pJexpress915 with TEM36 |
| | | | | | | | | insert cloned in Xhol/Notl |
| Nucleotide | 12 | CTXM16 | AAK32961 | Escherichia coli | E. coli./ | 900 | bp | Sequence of the insert |
| | | | | | P. pastoris | | | CTXM16 cloned in |
| | | | | | | | | Xhol/Notl |
| Nucleotide | 13 | TEM36-G₆- | Not available | Artificial | Pichia | 1680 | bp | Sequence of the insert |
| | | CTXM16 | | | pastoris | | | TEM36-G₆-CTXM16 cloned |
| | | | | | | | | in Xhol/Notl |
| Nucleotide | 14 | CTXM16- | Not available | Artificial | Pichia | 1634 | bp | Sequence of the insert |
| | | G₆-TEM36 | | | pastoris | | | CTXM16-G₆-CTXM16 |
| | | | | | | | | cloned in Xhol/Notl |
| Nucleotide | 15 | TEM36- | Not available | Artificial | Pichia | 1648 | bp | Sequence of the insert |
| | | G(EAAAK)₂- | | | pastoris | | | TEM36-G(EAAAK)2- |
| | | CTXM16 | | | | | | CTXM16 cloned in |
| | | | | | | | | Xhol/Notl |

| Nucleotide | 16 | CTXM16 | AAK32961 | Escherichia coli | E. coli | 870 | Insert Ndel/Hindlll coding for |
|---|---|---|---|---|---|---|---|
| | | | | | | | CTX-M16 in the vector |
| | | | | | | | pET26b+ |
| Nucleotide | 17 | AAC-6′-lb-cr | ABC17627.1 | Escherichia coli | E. coli | 627 | Insert Ndel/Hindlll coding for |
| | | | | | | | AAV in the vector pJ404 |
| Nucleotide | 18 | AAC-H6- | Not available | Artificial | E. coli | 1416 | Fusion AAC-6′-lb-cr with |
| | | CTXM16 | | | | | CTX-M16 with a Tag |
| | | | | | | | polyhistidine type linker |
| Nucleotide | 19 | EreB | P05789.1 | Escherichia coli | E. coli | 1287 | Insert Ndel/Hindlll coding for |
| | | | | | | | AAC in the vector pET26b+ |
| Nucleotide | 20 | EreB-H6- | Not available | Artificial | E. coli | 2076 | Fusion EreB with TEM36 |
| | | TEM36 | | | | | with a Tag polyhistidine type |
| | | | | | | | linker |
| Protein | 21 | AAC-H6- | Not available | Artificial | E. coli | 468 | Fusion AAC-6′-lb-cr with |
| | | CTXM16 | | | | | CTX-M16 with a Tag |
| | | | | | | | polyhistidine type linker |
| Protein | 22 | EreB-H6- | Not available | Artificial | E. coli | 688 | Fusion EreB with TEM36 |
| | | TEM36 | | | | | with a Tag polyhistidine type |
| | | | | | | | linker |

### EXAMPLE 1

### Hybrid Protein Molecule Consisting of the Fusion of TEM-36 and CTX-M16 by a Flexible Linker

The first embodiment of the present invention describes the fusion of IR-TEM (SEQ lD1); (Chaibi, Sirot et al., 1999)) with a cefotaximase (SEQ lD2; (Bonnet, Dutour et al., 2001)) via a flexible polyglycine linker (6 glycine residues in this example) and giving rise to a hybrid protein (SEQ lD8) able to hydrolyse even amoxicillin in the presence of clavulanic acid as well as ceftriaxone.

The nucleotide sequences encoding the proteins TEM-36 (SEQ lD1) and CTX-M16 (SEQ lD2) were commercially obtained by gene synthesis in an expression vector for Pichia pastoris (https://www.dna20.com/services/gene-synthesis?gclid=CPnQIp3c9r0CFaoewwodnREAlg). The nucleotide sequence of TEM-36 inserted into the expression vector corresponds to SEQ lD 11 and the nucleotide sequence of CTX-M16 inserted in the expression vector corresponds to SEQ lD 12. These sequences were then amplified by PCR (95° C. 30 sec-25 cycles [95° C. 30 sec-TM° C. 45 sec-72° C. 1 min]-72° C. 5 min-4° C.) using a high fidelity DNA polymerase (Pfu, Promega) and partially complementary primers and the protein linker constituent (GGGGGG in this example) as indicated in Table 2. TEM-36 was amplified using the pair of TEM36-F and TEM36-6G-R primers with a TM of 57° C. CTXM16 was amplified using the pair of 6G-CTXM16-F and CTXM16-R primers with a TM of 57° C. The constituent sequences of the linker GGGGGG are indicated in bold type and underlined in Table 2.

TABLE 2
List of primers used to construct the hybrid
sequence and protein TEM-36/CTX-M16 to a rigid linker.

| | ID SEQ | | |
|---|---|---|---|
| Primer name | No. | Sepuence5′-3′ | Host |
| TEM36-F | 23 | GAGGGTGTCTCTCTCGAG | Pichia |
| | | | pastoris |
| TEM36-6G-R | 24 | TCCACCTCCACCTCCTCCCCAATGTTTGATTAGGGA | Pichia |
| | | | pastoris |
| 6G-CTXM16-F | 25 | GGAGGTGGAGGTGGACAGACGTCAGCCGTGCAGCAAAAG | Pichia |
| | | | pastoris |
| CTXM16-R | 26 | GCTAGGCGGCCGCTTTTACAAACCTTCAGC | Pichia |
| | | | pastoris |

The two PCR fragments thereby obtained were hybridised at a TM of 55° C. and supplemented during 5 cycles of PCR without primers (95° C. 30 sec-55° C. 45 sec-72° C. 2 min15) using a high fidelity DNA polymerase (Pfu, Promega). The complete sequence encoding the hybrid protein was re-amplified after the addition of the external primers (TEM36-F+CTXM16-R). This embodiment of fusions by PCR is schematically presented in FIG. 3.

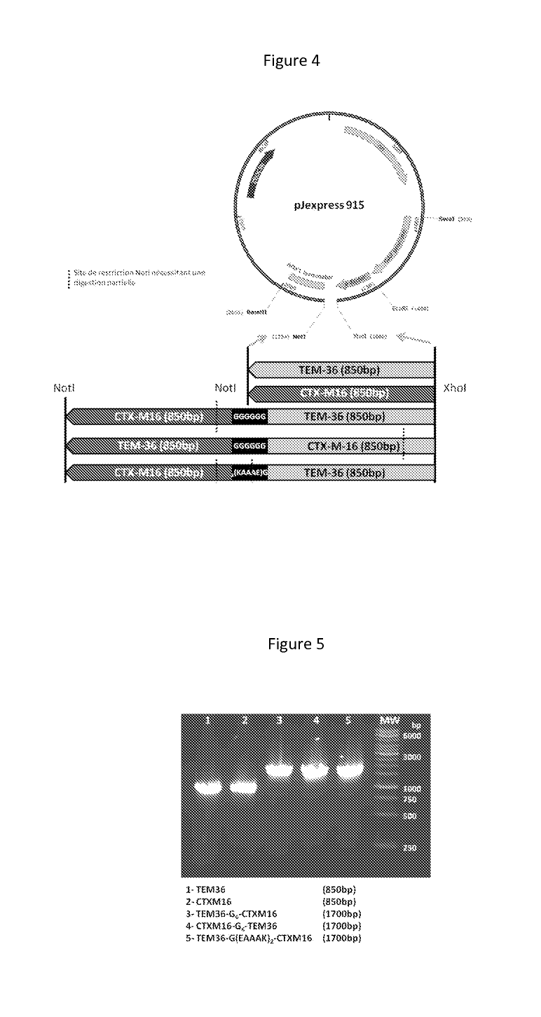

The TEM36-GGGGGG-CTXM16 fusion thereby produced was cloned with restriction enzymes Xhol and Notl in the pJexpress915 vector (expression Pichia pastoris) (FIG. 4). The sequence of the hybrid construction was verified in both directions and corresponds to the fusion described. FIG. 5 summarises the PCR fragments obtained with the primers pJexpress915-SEQ-F and pJexpress915-SEQ-R.

The vectors pJexpress915 expressing TEM-36, CTX-M16 and the fusion TEM36-GGGGGG-CTXM16 were amplified in Escherichia coli DH10B by maintaining a Zeocine (Invitrogen) selection pressure of 25 μg/ml on salt-depleted LB medium (Yeast extract 5 g/l; Tryptone 10 g/l; NaCl 5 g/l pH=7.5). To transform Pichia pastoris, 2 μg of linearized vector with the Swa l enzyme were electroporated into 60 μl of competent cells by a shock at 1500 V. The transformed cells are taken up in 1 ml of cold Sorbitol 1 M and put into culture for 2 hours at 30° C. prior to spreading (for 200 μl) on YPD-agar medium (Yeast extract 10 g/l; Peptone 20 g/l; Dextrose 20 g/l; agar 15 g/l-Phosphate 10 mM pH=6.8) containing 100 to 400 μg/ml of Zeocin and nitrocefin (20 μg/ml).

Expression in Pichia pastoris from the vector pJexpress915 leads to a secretion of proteins of interest, which are found in the culture medium. On YPD-agar plates containing nitrocefin, the secreted beta-lactamases induce a red hydrolysis halo (λ=486 nm) around the colonies that is representative of the level of expression. After 48 hours of incubation, the transformants with the best expression for each construct was inoculated in Sterlin tube containing 5 ml of YPD and 100 μg/ml of Zeocin and grown for 48 hours at 30° C. and stirred at 200 rpm.

The pre-cultures are then inoculated in 400 ml (200 ml per baffled Erlenmeyer flask) of fresh YPD medium without antibiotic selection pressure with an initial optical density of 0.2 at 600 nm. The culture is left for 48 hours at 30° C. under stirring at 100 rpm. After centrifugation at 10,000×g for 15 minutes at 4° C., the culture medium (supernatant) is stored at 4° C. in the presence of benzamidine (1 mM final). The proteins of the culture medium are then concentrated 10 times by tangential filtration through a 10 kDa membrane (Vivaflow 10 module, Sartorius) to a volume of 40 ml, then to 10 ml by ultrafiltration on 10 kDa membrane (Centricon, Millipore). The 10 ml are injected on a size exclusion chromatography column (Superdex G75 for the TEM-36 and CTX-M16 proteins, Superdex G200 for melting; 26*60 columns GE Healthcare), and eluted in 10 mM of sodium phosphate buffer pH=7.0 by 1 ml fraction and with a flow rate of 1 ml/min.

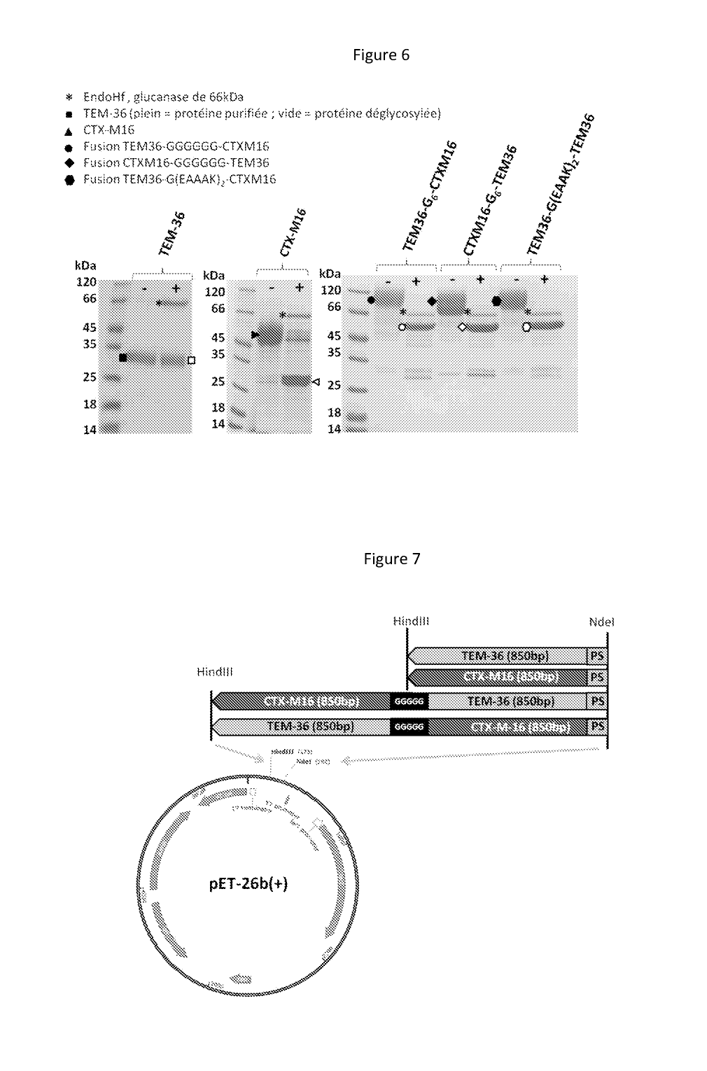

The fractions with the highest activity on nitrocefin (VWR) and the best purity (SDS-PAGE) is combined and concentrated to about 0.1-0.5 mg/ml. The protein content of the samples is measured by BCA (Pierce), absorbance at 280 nm and verification on SDS-PAGE gel. FIG. 6 provides the results.

For CTX-M16 and the fusion, which have N-glycosylation sites, the glycosylation is verified by cleavage of the sugars with EndoHf enzyme (New EnglandBiolabs) according to the manufacturer's recommendations (overnight at 37° C. in non-denaturant conditions). The proteins produced and purified are confirmed by tryptic digestion and MALDI-TOF mass spectrometry analysis.

As indicated in FIG. 6, the TEM-36 protein is not glycosylated and its size is compatible with the expected size of 28 kDa, as opposed to the CTX-M16 proteins and the different fusions. The apparent molecular weight of the latter on SDS-PAGE gel is higher (45 kDa versus 28 kDa and >66 kDa versus 56 kDa for CTX-M16 and the fusions respectively. Cutting with EndoHf provides proteins with the expected molecular weight (28 kDa and 56 kDa for CTX-M16 and the respective fusions), thereby confirming that these proteins are glycosylated.

The purified proteins were tested on different beta-lactams (amoxicillin (Apollo Scientific), Augmentin® (GlaxoSmithKline) and Ceftriaxone (Rocéphine®, Roche)). The assays are carried out at pH-STAT (Titrino 2.5, Metrohm) in a reaction volume of 25 ml at 37° C. and pH=7.0. The substrates are prepared at 4 g/l in a 0.3 mM Tris buffer, 150 mM NaCl. The enzyme hydrolysis of the beta-lactam nuclei releases an acid and induces a drop in the pH. The principle of the assay is to compensate for the acidification by the addition of 0.1 N sodium hydroxide so as to remain at pH=7.0. In these conditions, one unit corresponds to 1 μmole of sodium hydroxide added per minute, that is, one μmole of beta-lactam hydrolysed per minute. Table 3 below lists the specific activities measured for each protein on the three substrates (mean of 6 replicates).

TABLE 3
Specific activities of hybrid protein TEM-
36/CTX-M16 with a flexible linker and their constituent
enzymes produced and purified in Pichia pastoris

| | Specific activity (U/mg) | | |
|---|---|---|---|
| | Substrate | | |
| Proteins | Amoxicillin | Augmentin ® | Ceftriaxone |
| TEM-36 | 816 ± 90 | 97 ± 74 | 0 |
| CTX-M16 | 31 ± 2 | 0 | 306 ± 44 |
| TEM36-GGGGGG- | 695 ± 98 | 42 ± 33 | 114 ± 44 |
| CTXM16 | | | |

These results show that the fusion between TEM-36 and CTX-M16 generates a hybrid protein with an activity both on an aminopenicillin even in the presence of a beta-lactamase inhibitor and a third generation cephalosporin. Since both activities are described as antagonist in the literature (Ripoll, Baquero et al., 2011), there is no known natural enzyme with such high catalytic constants on these two substrates.

### EXAMPLE 2

### Hybrid Protein Molecule Consisting of the Fusion of CTX-M16 and TEM-36 by a Flexible Linker

A second embodiment of the present invention describes the fusion of a cefotaximase (SEQ lD2; (Bonnet, Dutour et al., 2001)) with an lR-TEM (SEQ lD1; (Chaibi, Sirot et al., 1999)) via a flexible linker polyglycine (6 glycine residues in this example) and giving rise to a hybrid protein (SEQ lD9) able to hydrolyse amoxicillin even in the presence of clavulanic acid as well as ceftriaxone.

This embodiment is identical that described in Example 1 with the exception of the PCR primers used to amplify sequences CTXM16 and TEM-36.

CTXM-16 was amplified using a pair of primers CTX16-F and CTXM16-6G-R with a TM of 58° C. TEM-36 was amplified using the pair of primers 6G-TEM36-F and TEM36-R with a TM of 58° C. The constituent sequences of the linker GGGGGG are indicated in bold type and underlined in Table 4.

TABLE 4
List of primers used to construct and sequence
the hybrid protein CTX-M16/TEM-36 with a flexible
linker.

| | ID SEQ | | |
|---|---|---|---|
| Primer name | No. | Sepuence5′-3′ | Host |
| CTXM16-F | 27 | GAGGGTGTCTCTCTCGAG | Pichia |
| | | | pastoris |
| CTXM16-6G-R | 28 | TCCTCCTCCTCCTCCTCCCAAACCTTCAGCTATGATCCG | Pichia |
| | | | pastoris |
| TEM36-6G-F | 29 | GGAGGAGGAGGAGGAGGACACCCTGAGACACTTGTCAAG | Pichia |
| | | | pastoris |
| TEM36-R | 30 | TTGAGCGGCCGCCCCTCA | Pichia |
| | | | pastoris |

The two PCR fragments thereby obtained were hybridised at a TM of 55° C. and supplemented during 5 cycles of PCR without primers (95° C. 30 sec-55° C. 45 sec-72° C. 2 min15) using a high-fidelity DNA polymerase (Pfu, Promega). The complete sequence encoding the hybrid protein was re-amplified after the addition of the external primers (CTXM16-F+TEM-36-R).

The conditions used for the expression and purification of the hybrid protein CTXM16-GGGGGG-TEM36 were strictly identical to those described in Example 1. The hybrid protein thereby obtained is also glycosylated as shown in FIG. 6 and combines a persistent penicilinase activity in the presence of clavulanic acid with a cefotaximase activity.

As in the case of Example 1, no natural protein presents the specific activities described in Table 5 on both substrates amoxicillin/clavulanic acid and ceftriaxone.

TABLE 5
Specific activities of hybrid protein CTX-
M16/TEM-36 with a rigid linker and their constituent
enzymes produced and purified in Pichia pastoris

| | Specific activity (U/mg) | | |
|---|---|---|---|
| | Substrate | | |
| Proteins | Amoxicillin | Augmentin ® | Ceftriaxone |
| TEM-36 | 816 ± 90 | 97 ± 74 | 0 |
| CTX-M16 | 31 ± 2 | 0 | 306 ± 44 |
| TEM36-GGGGGG- | 890 ± 83 | 42 ± 14 | 123 ± 35 |
| CTXM16 | | | |

### EXAMPLE 3

### Hybrid Protein Molecule Consisting of the Fusion of TEM-36 and CTX-M16 by a Rigid Linker

The third embodiment of the present invention describes the fusion of an IR-TEM (SEQ lD1; (Chaibi, Sirot et al., 1999)) with a cefotaximase (SEQ lD2; (Bonnet, Dutour et al., 2001)) via a rigid protein linker (motif G(EAAAK)₂ in this example) and giving rise to a hybrid protein (SEQ lD10) able to hydrolyse amoxicillin even in the presence of clavulanic acid as well as ceftriaxone.

This embodiment is identical that described in Example 1 with the exception of the PCR primers used to amplify the sequences CTXM16 and TEM-36. TEM-36 was amplified using the pair of primers TEM36-F and TEM36-G(EAAAK)₂ with a TM of 58° C. CTXM16 was amplified using the pair of primers CTXM16-G(EAAAK)₂-F and CTXM16-R with a TM of 58° C. The constituent sequences of the linker G(EAAAK)₂ are indicated in bold type and underlined in Table 6.

TABLE 6
List of primers used to construct and sequence
the hybrid protein CTX-M16 and TEM-36 with a rigid
linker.

| | ID SEQ | | |
|---|---|---|---|
| Primer name | No. | Sepuence5′-3′ | Host |
| TEM36-F | 23 | GAGGGTGTCTCTCTCGAG | Pichia |
| | | | pastoris |
| CTXM16-R | 26 | GCTAGGCGGCCGCTTTTACAAACCTTCAGC | Pichia |
| | | | pastoris |
| TEM36- | 31 | TGCTGCCTCTTTAGCGGCCGCCTCTCCCCAATGTTTGATTAGGGA | Pichia |
| G(EAAAK)₂-R | | | pastoris |
| CTXM16- | 32 | GCCGCTAAAGAGGCAGCAGCAAAACAGACGTCAGCCGTG | Pichia |
| G(EAAAK)₂-F | | | pastoris |

The two PCR fragments thereby obtained were hybridised at a TM of 55° C. and supplemented during 5 cycles of PCR without primers (95° C. 30 sec-55° C. 45 sec-72° C. 2 min15) using a high-fidelity DNA polymerase (Pfu, Promega). The complete sequence encoding for the hybrid protein was re-amplified after the addition of external primers (TEM36-F+CTXM16-R).

The conditions used for the expression and purification of the hybrid protein TEM36-G(EAAAK)₂-TEM36 were strictly identical those described in Example 1. The hybrid protein thereby obtained is also glycosylated (FIG. 6) and combines a persistent penicillinase activity in the presence of clavulanic acid with a cefotaximase activity.

TABLE 7
Specific activities of hybrid protein CTX-
M16/TEM-36 with a flexible linker and their constituent
enzymes produced and purified in Pichia pastoris

| | Specific activity (U/mg) | | |
|---|---|---|---|
| | Substrate | | |
| Proteins | Amoxicillin | Augmentin ® | Ceftriaxone |
| TEM-36 | 816 ± 90 | 97 ± 74 | 0 |
| CTX-M16 | 31 ± 2 | 0 | 306 ± 44 |
| TEM36-GGGGGG- | 890 ± 176 | 34 ± 6 | 141 ± 55 |
| CTXM16 | | | |

As in Examples 1 and 2, no natural protein presents the specific activities described in Table 7 on both substrates amoxicillin/clavulanic acid and ceftriaxone.

### EXAMPLE 4

### Non-Glycosylated Hybrid Protein Molecules Consisting of the Fusion of TEM-36 and CTX-M16 (and Vice-Versa) by a Flexible Linker

The coding sequences for TEM-36 (SEQ lD1) and CTXM16 (SEQ lD2) were commercially obtained by gene synthesis (https://www.dna20.com/services/gene-synthesis?gclid=CPnQlp3c9r0CFaoewwodnREAlg) in a periplasmic expression vector for E. coli. The nucleotide sequences were obtained in phase with the reading frame encoding a periplasmic targeting peptide (MSIQHFRVALIPFFAAFCLPVFA) with an Ndel restriction site at the initiating methionine and a Hindlll restriction site after the stop codon. Gene fragments Ndel/Hindlll were then sub-cloned in the pET-26b(+) vector (Invitrogen).

As shown in FIG. 3, we created two hybrid protein molecules by overlap PCR. The nucleotide sequence of TEM-36 and CTX-M16 were amplified by PCR (95° C. 30 sec-25 cycles [95° C. 30 sec-TM° C. 45 sec-72° C. 1 min]-72° C. 5 min-4° C.) using a high-fidelity DNA polymerase (Pfu, Promega) and partially complementary primers constituting the linker protein (GGGGGG in this example). TEM-36 was amplified with a TM of 55° C. using either the primer pair T7-F and TEM36-G6-R-co or the primer pair TEM36-G6-F-co and T7-R. CTXM16 was amplified with a TM of 55° C. using the primer pair CTXM16-G6-F-co and T7-R or the primer pair CTXM16-G6-R-co and T7-F. The constituent sequences of the linker GGGGGG are indicated in bold type and underlined in Table 8.

TABLE 8
List of primers used to construct and sequence
the hybrid protein TEM-36/CTX-M16 with a flexible
linker.

| | ID SEQ | | |
|---|---|---|---|
| Primer name | No. | Sepuence5′-3′ | Host |
| T7-F | 33 | TAATACGACTCACTATAGGGGAAT | E. coli |
| TEM36_36G_R_co | 34 | TCCTCCTCCTCCTCCTCCCCAATGTTTAATCAGGCT | E. coli |
| CTXM16-G6_F_co | 35 | GGAGGAGGAGGAGGACAGACGTCAGCCGTGCAGCAAAAG | E. coli |
| CTXM16-G6_R_co | 36 | TCCTCCTCCTCCTCCTCCCAAACCTTCAGCTATGATCCG | E. coli |
| TEM36-6G-F | 37 | GGAGGAGGAGGAGGAGGACACCCTGAGACACTTGTCAAG | E. coli |
| T7-R | 38 | CTAGTTATTGCTCAGCGGTGG | E. coli |

The two PCR fragments thereby obtained (TEM36-G6+G6-CTXM16 and CTXM16-G6+G6-TEM36) were hybridised at a TM of 55° C. and supplemented during 5 cycles of PCR without primers (95° C. 30 sec-53° C. 45 sec-72° C. 2 min15) using a high-fidelity DNA polymerase (Pfu, Promega). The complete sequence encoding for the hybrid protein was re-amplified after the addition of external primers (T7-F+T7-R) operating at a TM of 55° C.

The fusions TEM36-GGGGGG-CTXM16 and CTX-M16-GGGGGG-TEM36 thereby produced were cloned with the Ndel and Hindlll restriction enzymes in a pET26b+ vector (expression in E. coli) (FIG. 7).

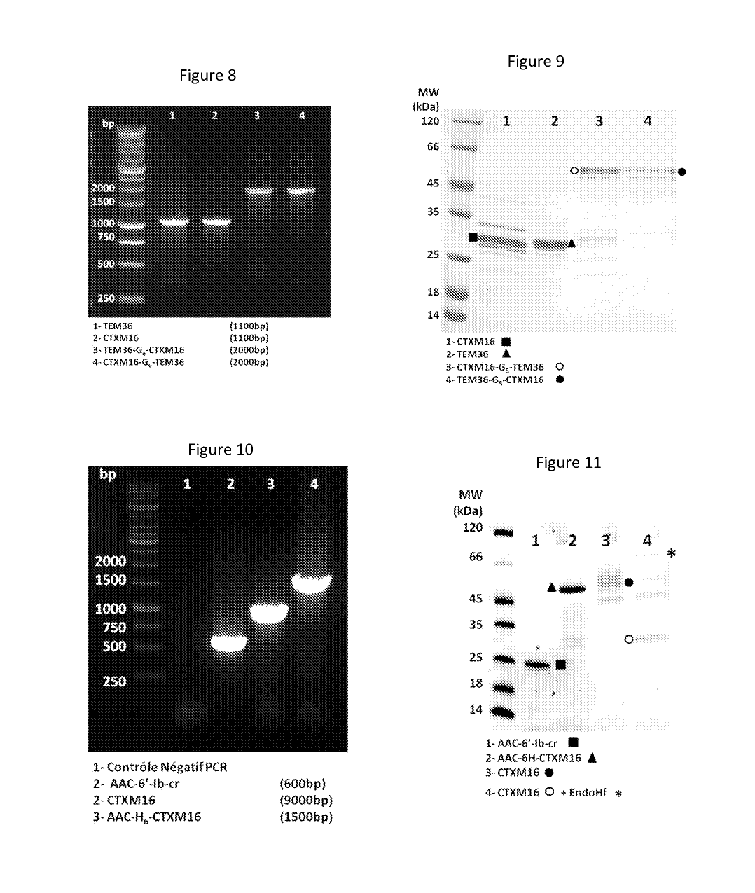

The sequences of the hybrid constructs were verified in directions and revealed that the linker of the 2 hybrid protein molecules actually consist of only 5 glycines. A more favourable nucleic rearrangement probably occurred at the time of the PCR hybridations. FIG. 8 sums up the PCR fragments obtained with the primers T7-F and T7-R.

The pET-26b(+) expression vectors expressing TEM-36 (SEQ lD1), CTX-M16 (SEQ lD2) and the fusions TEM36-G₅-CTXM16 and CTXM16-G₅-TEM36 were amplified in Escherichia coli DH10B by maintaining a Kanamycin (Euromedex) selection pressure at 50 μg/ml on LB medium (Yeast extract 5 g/l; Tryptone 10 g/l; NaCl 10 g/l pH=7). The expression strain BL21(DE3) pLysS was transformed with the expression vectors by heat shock. The transformed cells were spread on LB-agar medium (Yeast extract 5 g/l; Tryptone 10 g/l; NaCl 10 g/l; Agar 15 g/l pH=7) containing 50 μg/ml of Kanamycin.

The expression in E. coli from the pET-26b(+) vector leads to an addressing of the proteins of interest in the periplasmic compartment of bacteria where they are functional. The fusions created as described in Example were evaluated in 2 complementary ways: (i) by biochemically characterising the partially purified proteins and (ii) by comparing the minimum inhibitory concentrations (MIC) of different b-lactams (amoxicillin, Augmentin and Rocéphine) on the expression strains.

Purification of Hybrid Protein Molecules and Their Component in E. Coli:

The transformed bacteria are grown in 100 ml of LB+50 μg/ml of Kanamycin at 37° C. and with stirring at 200 rpm until saturation. The pre-cultures are then inoculated at 1/40^{th} in 1 L of LB medium+50 μg/ml Kanamycin. When the OD600 nm reached a value of 0.6 (about 2 hours), the protein production is induced by the addition of 0.5 mM final IPTG and continues for 16 hours at 20° C. with stirring at 200 rpm. The cells are centrifuged at 5,000×g for 15 minutes at 4° C. and the sediment immediately taken up in an osmolysis buffer to break the outer membrane and recover the periplasma. The cells are taken up with 1 ml of buffer (Phosphate 100 mM, Sucrose 500 mM, EDTA 1 mM pH=7.0) for 120 Units of OD600 nm and incubated for several minutes with vortex homogenisation (protocol adapted from (Schlegel, Rujas et al. 2013)). After centrifugation at 12,000×g for 20 minutes at 4° C., the supernatant containing the periplasmic proteins is concentrated on Amicon Ultra (15 ml, 10 kDa, Millipore) until the volume does not exceed 10 ml. The totality of the proteins is injected on an exclusion chromatography column (Superdex G75, GE Healthcare) and the proteins are eluted in phosphate buffer (Phosphate 10 mM, NaCl 100 mM pH=7.0) per 1 ml fraction and with a flow of 1 ml/min. The fractions with the highest activity on nitrocefin (VWR) and the best purity (SDS-PAGE) are combined and concentrated to about 0.1-0.5 mg/ml. The protein content of the samples is measured by absorbance at 280 nm and verification on SDS-PAGE gel. FIG. 9 presents the partially purified proteins as obtained before biochemical characterisation.

The enzyme activities of the purified proteins were measured on different beta-lactams (amoxicillin (Apollo Scientific), Augmentin® (GlaxoSmithKline) and Ceftriaxone (Rochéphine®, Roche)) as described in the above examples and the results are compiled in Table 9 below.

TABLE 9
Specific activities of hybrid protein
molecules TEM36-G₅-CTXM16 and CTXM16-G₅-TEM36 (flexible
linker) and their constituent enzymes produced and
purified in Escherichia coli BL21(DE3) pLysS.

| | Specific activity (U/mg) | | |
|---|---|---|---|
| | Substrate | | |
| Proteins | Amoxicillin | Augmentin ® | Ceftriaxone |
| TEM-36 | 3593 ± 87 | 385 ± 31 | 1 ± 1 |
| CTX-M16 | 21 ± 1 | 0 | 158 ± 14 |
| TEM36-GGGGGG- | 633 ± 99 | 24 ± 5 | 138 ± 32 |
| CTXM16 | | | |
| CTXM16-GGGGG- | 566 ± 32 | 17 ± 5 | 103 ± 3142 |
| TEM36 | | | |

As in Examples 1 to 3, no natural protein presents the specific activities described in Table 9 on both substrates amoxicillin and ceftriaxone.

Resistance of Strains of E. Coli Expressing the Hybrid Protein Molecules to Various Beta-Lactams:

The transformed bacteria (expressing TEM36, CTXM16 or the fusions TEM36-G5-CTXM16 and CTXM16-G5-TEM36) or the empty strain (BL21(DE3)pLysS) are put in culture in 5 ml of LB+50 μg/ml of Kanamycin when necessary at 37° C. and stirring at 200 rpm until saturation. The pre-cultures are then inoculated at 1/40^{th} in 5 ml of LB+50 μg/ml Kanamycin medium and when the OD600 reaches a value of 0.6 (about 2 hours), the production of protein is induced by the addition of final IPTG 1 mM and continues for 1.5 hours at 37° C. with stirring at 200 rpm.

The cells are normalised at 108 cfu/ml and diluted in cascade to 10⁷, 10⁶, 10⁵, 10⁴ cfu/ml and 5 μl of each suspension are deposited on an LB-Agar plate containing increasing concentrations of antibiotics (Amoxicillin 0, 2, 4, 8, 16, 32, 64, 128, 256, 512, 1024 μg/ml; Augmentin 0, 1, 2, 4, 8, 16, 32, 64, 128, 256, 512 μg/ml and Rocéphine 0, 0.5, 1, 2, 4, 8, 16, 32, 64, 128, 256, 512 μg/ml).

The dishes were incubated at 37° C. overnight and the results were recorded the following day. The MICs correspond to the lowest concentration of antibiotic at which the highest inoculum doesn't grow.

The data is provided in Table 10 below.

TABLE 10
MICs of beta-lactam antibiotics for strains
of E. coli expressing the hybrid protein molecules
TEM36-G₅-CTXM16 and CTXM16-G₅-TEM36 (flexible linker)
and their constituent enzymes.

| | Antibiotics | | |
|---|---|---|---|
| | MIC (μg/ml) | | |
| Strain | Amoxicillin | Augmentin | Rocephine |
| BL21(DE3)pLysS | <0.5 | <0.5 | <0.5 |
| BL21(DE3)pLysS + TEM36 | >1024 | 512 | <0.5 |
| BL21(DE3)pLysS + CTXM16 | >1024 | 4 | 512 |
| BL21(DE3)pLysS + TEM36-G₅- | >1024 | 128 | 64 |
| CTXM16 | | | |
| BL21(DE3)pLysS + CTXM16- | >1024 | 128 | 128 |
| G₅-TEM36 | | | |

The E. coli cells expressing the hybrid protein molecules as described in Example 4 present a multidrug resistance phenotype to aminopenicillins (with or without inhibitors such as clavulanic acid) and 3^{rd} generation cephalosporins such as ceftriaxone. No natural bacteria strain has so far been described in the literature with such a phenotype.

### EXAMPLE 5

### Hybrid Protein Molecule Consisting of the Fusion of CTX-M16 and AAC-6′-lb-cr by a Poly-Histidine Linker

The nucleotide sequences encoding the CTX-M16 (SEQ lD2) and AAC-6′-lb-cr proteins (hereafter called AAC) (SEQ lD4) were commercially obtained by gene synthesis in an expression vector for E. coli, respectively pJexpress411 (KanR) and pJ404(AmpR). The nucleic sequence of CTX-M16 inserted in the vector pJexpress411 corresponds to SEQ lD16 and the nucleic sequence of AAC inserted in the vector pJ404 corresponds to SEQ lD12. These sequences were then amplified by PCR (95° C. 30 sec-25 cycles [95° C. 30 sec-TM° C. 45 sec-72° C. 1 min]-72° C. 5 min-4° C.) using a high-fidelity DNA polymerase (Pfu, Promega) and partially complementary primers constituent of the protein linker (HHHHHH in this example) as indicated in Table 11. CTX-M16 was amplified using the primer pair 6H-CTX-F and T7R with a TM of 62° C. ACC was amplified using the primer pair AAC-F_coli and AAC-6H-R with a TM of 50° C. The constituent sequences of the linker HHHHHH are indicated in bold type underlined in Table 11.

TABLE 11
List of primers used to construct and
sequence the hybrid protein AAC-H6-CTXM16 with a
polyhistidine linker.

| | ID SEQ | | |
|---|---|---|---|
| Primer name | No. | Sepuence5′-3′ | Host |
| AAC-F_coli | 39 | GAAGGAGATATACATATGAGCAACGCT | E. coli |
| AAC-6H-R | 40 | GTGGTGATGATGGTGGTGCGC | E. coli |
| H6-CTX-F | 41 | CACCATCATCACCACCAGACGTCAGCCGTGCAGCAAAAG | E. coli |
| T7-R | 38 | CTAGTTATTGCTCAGCGGTGG | E. coli |

The two PCR fragments thereby obtained were hybridised at a TM of 62° C. and supplemented during 5 cycles of PCR without primers (95° C. 30 sec-62° C. 45 sec-72° C. 2 min15) using a high-fidelity DNA polymerase (Pfu, Promega). The complete sequence encoding for the hybrid protein was re-amplified after addition of the external primers (AAC-F_coli+T7R). This embodiment of fusions by PCR is schematically presented in FIG. 3. The inserts encoding for AAC, CTXM16 and the fusion AAC-6H-CTXM16 were loaded on 1% agarose gel to illustrate their respective size (FIG. 10).

The fusion AAC-6H-CTXM16 thereby produced was cloned with Ndel and HinDlll restriction enzymes in the pET26b+ vector (E. coli expression) according to the principle illustrated in FIG. 7.

The sequence of the hybrid construction was verified in both directions and corresponds to the fusion described.

The pJ404-AAC expression vectors expressing AAC and pET-AAC-6H-CTXM16 expressing the fusion AAC-H6-CTXM16 were transformed in Escherichia coli BL21(DE3)pLysS by maintaining an 100 μg/ml Ampicillin (Euromedex) and 50 μg/ml Kanamycin (Euromedex) selection pressure on LB medium (Yeast extract 5 g/l; Tryptone 10 g/l; NaCl 10 g/l pH=7.5. The expression of AAC as well as the AAC-6H-CTX fusion in E. coli from vector pJ404 results in inclusion bodies for the proteins of interest.

For each protein, 1 L of LB is seeded at 1/40^{th} from a saturated pre-culture and then grown at 37° C. with stirring at 200 rpm until an OD (600 nm) of about 0.4-0.6. The cultures are induced for 4 hours at 37° C. and 200 rpm by the addition of 0.5 mM final IPTG. At the end of production, the cells are centrifuged and the sediment is taken up at 40 ml/L of culture in lysis buffer (10 mM Tris-HCl, 150 mM NaCl, 1 mM EDTA, 0.1% Triton X100 pH=8.0 and 0.25 mg/ml lysozyme) and then frozen at −80° C. The cells are thawed out and lysed for 45 minutes at ambient temperature in the presence of MgSO₄ (20 mM) and DNAse (10 μg/ml). The lysate is centrifuged (30 min at 12,000×g at 4° C.) and the sediment containing the inclusion bodies of the proteins of interest (AAC and AAC-6H-CTXM16) is taken up in buffer A (10 mM Phosphate, 150 mM NaCl, 10 mM Imidazole, 8 M Urea pH=8.0). The proteins are purified by Nickel affinity chromatography and eluted with a gradient of buffer B (10 mM Phosphate, 150 mM NaCl, 500 mM Imidazole, 8 M Urea pH=8.0).

The fractions containing the proteins of interest are mixed, incubated for 1 hour at 4° C. in the presence of 1 mM DTT, then re-natured by 3 successive dialyses in 10 mM Phosphate buffer, 150 mM NaCl pH=8. The proteins are clarified by centrifugation then concentrated to a volume not exceeding 10 ml by ultrafiltration through a 10 kDa membrane (Centricon, Millipore). The proteins are injected onto a size exclusion chromatography column (Superdex G200; columns 26*60 GE Healthcare) and eluted in 10 mM Phosphate buffer, 150 mM NaCl pH=8.0 by 1 ml fraction with a flow of 1 ml/min.

The fractions with the highest activity on Nitrocefin (VWR) and the best purity (SDS-PAGE) are combined and concentrated to about 0.5 mg/ml. The protein content of the samples is measured by BCA (Pierce), absorbance at 280 nm and verification on SDS-PAGE gel. FIG. 11 presents the results of the purified proteins obtained.

The purified proteins were tested on different antibiotics: Ceftriaxone (RocéphineED, Roche) for the beta-lactams and kanamycin for the Aminoglycosides. The assays on ceftriaxone were carried out at pH-STAT (Titrino 2.5, Metrohm) in a reaction volume of 25 ml at 37° C. and pH=7.0. The substrates are prepared at 4 g/l in a 0.3 mM Tris buffer, 150 mM NaCl. The enzyme hydrolysis of the beta-lactam nuclei releases an acid and results in a drop in the pH. The principle of the assay is to compensate this acidification by the addition of 0.1 N sodium hydroxide so as to remain at pH=7.0. In these conditions, one unit corresponds to 1 μmol of sodium hydroxide added per minute, that is one μmol of beta-lactam hydrolyzed per minute.

The activity of acety-transferase is measured by an indirect colorimetric assay with acetyl-CoA (Sigma-Aldrich) as acetyl group donor, Kanamycin as acceptor and Elleman reagent (DTNB, Sigma-Aldrich) to titrate the CoEnzymeA reduced (—SH) molecules released during the enzyme reaction [Ref]. The reaction takes place on a microtitration plate with increasing quantities of purified enzymes, in a final volume of 200 μl and with concentrations of 500 μM Kanamycin, 500 μM AcetylCoA and 250 μM DTNB. In these experimental conditions, the ion TNB² absorbs at 412 nm with an ε(λ=412 nm) apparent 19 000 M⁻¹.well⁻¹ (well of a 96 well plate filled with 200 μl) and 1 unit corresponds to one nanomole of TNB² released per minute at 37° C.

Table 12 below sums up the specific activities measured for each protein on the three substrates (mean of 6 experiments, that is, n=6).

TABLE 12
Specific activities of hybrid protein AAC-H6-
CTX-M16 and their constituent enzymes

Specific activity (U/mg)
Substrate

| Proteins | Kanamycin | Cetriaxone |
|---|---|---|
| AAC | 10.2 ± 3.2 | 0 |
| CTX-M16 | 0 | 336 ± 5 |
| AAC-6H-CTXM16 | 14.2 ± 2.4 | 270 ± 36 |

These results show that the fusion between AAC-6′-lb-cr and CTX-M16 generates a hybrid protein able to hydrolyze a third generation cephalosporin and inactivate an aminoglycoside by acetylation. There is no known natural enzyme capable of inactivating an antibiotic in these two classes.

### EXAMPLE 6

### Non-Glycosylated Hybrid Protein Molecules Consisting of the Fusion of EreB and TEM36 by a Tag Polyhistidine Type Linker

The sequences encoding for TEM-36 (SEQ lD1) and EreB (SEQ lD5) were commercially obtained by gene synthesis in an expression vector for E. coli. The gene fragments Ndel/Hindlll were then sub-cloned in the pET-26b(+) vector (Invitrogen).

According to the principle described in FIG. 3, we produced a hybrid protein molecule EreB-H6-TEM36 by overlap PCR. The nucleotide sequence of TEM-36 and EreB were amplified by PCR (95° C. 30 sec-25 cycles [95° C. 30 sec-TM° C. 45 sec-72° C. 1 min]-72° C. 5 min-4° C.) using a high-fidelity DNA polymerase (Pfu, Promega) and partially complementary primers constitutive of the protein linker (histidine tag 6 in this example). TEM-36 was amplified with a TM of 65° C. using primer pair EreB6HTEM36_coli_F and T7_R. EreB was amplified with a TM of 65° C. using the primer pair EreB_coli_F and EreB6HTEM36_R. The constituent sequences of the HHHHHH linker are indicated in bold type and underlined in Table 13.

TABLE 13
List of primers used to construct and
sequence the hybrid protein EreB-H6-TEM-36.

| | ID | | |
|---|---|---|---|
| | SEQ | | |
| Primer name | No. | Sepuence5′-3′ | Host |
| EreB_coli_F | 39 | GATATACATATGCGTTTTGAAGAGTGG | E. coli |
| EreB6HTEM36_R | 40 | GTGATGGTGATGGTGGTGCTCATAAAC | E. coli |
| EreB6HTEM36_coli_F | 41 | CACCACCATCACCATCACCACCCGGAAACCCTGGTGAAAGTT | E. coli |
| T7-R | 38 | CTAGTTATTGCTCAGCGGTGG | E. coli |

The PCR fragments thereby obtained were hybridized at a TM of 55° C. and supplemented during 5 cycles of PCR without primers (95° C. 30 sec-55° C. 45 sec-72° C. 2 min15) using a high-fidelity DNA polymerase (Pfu, Promega). The complete sequence encoding for the hybrid protein was re-amplified after the addition of external primers (EreB_coli_F+T7-R) operating at a TM of 55° C.

The EreB-H6-TEM36 fusion thereby produced was cloned with the Ndel and Hindlll enzyme restrictions in the pET26b+ vector (expression in E. coli) (FIG. 7).

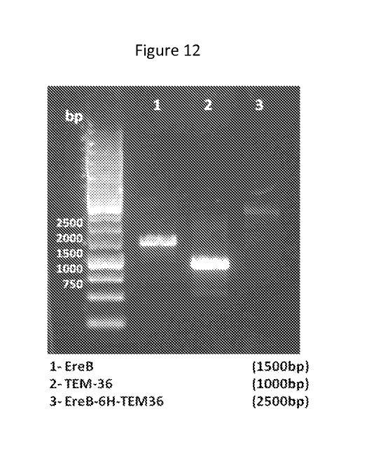

The sequence of hybrid construction was verified in both directions. FIG. 12 sums up the PCR fragments obtained with the T7-F and T7-R primers on the pET-TEM36, EreB and EreB-H6-TEM36 series.

The pET-26b(+) expression vectors expressing TEM-36 (SEQ lD1), EreB (SEQ lD2) and the fusion EreB-H6-TEM36 were amplified in Escherichia coli DH10B while maintaining a 50 μg/ml Kanamycin (Euromedex) selection pressure on LB medium (Yeast extract 5 g/l; Tryptone 10 g/l, NaCl 10 g/l pH=7). The expression strain BL21(DE3) pLysS was transformed with the expression vectors by heat shock. The transformed cells were spread on LB-agar medium (Yeast extract 5 g/l; Tryptone 10 g/l; NaCl 10 g/l; agar 15 g/l pH=7) containing 50 μg/ml of Kanamycin.

The functionality of the proteins of interest (TEM36, EreB and the fusion EreB-H6-TEM36) in E. coli was evaluated by measuring the resistance of the expression strains to different b-lactam (amoxicillin and augmentin) and macrolide (erythromycin) type antibiotics.

The transformed bacteria (expressing TEM36, EreB or EreB-H6-TEM36) or the empty strain (BL21(DE3)pLysS) are put in culture in 5 ml of LB+50 μg/ml of Kanamycin when necessary at 37° C. with stirring at 200 rpm until saturation. The pre-cultures are then inoculated at 1/40^{th} in 5 ml of LB+50 μg/ml Kanamycin medium and when the OD600 nm reaches a value of 0.6 (about 2 hours), the production of protein is induced by the addition of 1 mM final IPTG and continued for 1.5 hours at 37° C. with stirring at 200 rpm.

The cells are normalized to 108 cfu/ml and diluted in cascade to 10⁷, 10⁶, 10⁵, 10⁴ cfu/ml and 5 μl of each suspension are deposited on an LB-agar dish containing increasing concentrations of antibiotics (Amoxicillin 0, 2, 4, 8, 16, 32, 64, 128, 256, 512, 1024, 2048 μg/ml; Augmentin 0, 2, 4, 8, 16, 32, 64, 128, 256, 512, 1024 μg/ml and Erythromycin 0, 2, 4, 8, 16, 32, 64, 128, 256, 512, 1024 μg/ml).

The dishes are incubated at 37° C. over night and the results are recorded the next day. The MICs correspond to the lowest concentration of antibiotic at which the highest inoculum no longer grows.

The data are provided in Table 14 below.

TABLE 14
MICs of beta-lactam and macrolide antibiotics
for strains of E. coli expressing the hybrid protein
molecule EreB-H6-TEM36 and their constituent enzymes.

| | Antibiotics | | |
|---|---|---|---|
| | MIC (μg/ml) | | |
| Strain | Amoxicillin | Augmentin | Erythromycin |
| BL21(DE3)pLysS | <2 | <2 | 256 |
| BL21(DE3)pLysS + | >2048 | 512 | 256 |
| TEM36 | | | |
| BL21(DE3)pLysS + EreB | <2 | <2 | >1024 |
| BL21(DE3)pLysS + EreB- | 32 | 8 | >1024 |
| H6-TEM36 | | | |

The E. coli cells expressing the hybrid protein molecule as described in Example 6 presents a multidrug resistance phenotype to aminopenicillins (with or without inhibitors such as clavulanic acid) and macrolides such as erythromycin. No natural bacteria strain has so far been described in the literature with such a phenotype resulting from the expression of a single protein.

1. A hybrid protein molecule comprising two enzymes that inhibit the activity of at least one antibiotic, one of said enzymes is a beta-lactamase and the other said enzyme is a beta-lactamase, an enzyme that inhibits an aminoglycoside, an enzyme that inhibits an fluoroquinolone, an enzyme that inhibits a macrolide, an enzyme that inhibits a tetracycline, or an enzyme that inhibits a lincosamide, said enzymes being bonded together.

2. The hybrid protein molecule according to claim 1, comprising two beta-lactamases linked together.

3. The hybrid protein molecule according to claim 1, wherein the enzyme that inhibits an aminoglycoside is a phosphotransferase, a nucleotidyltransferase or an acetyltransferase.

4. The hybrid protein molecule according to claim 1, wherein the enzyme that inhibits a fluoroquinolone is an aminoglycoside N-acetyltransferase.

5. The hybrid protein molecule according to claim 1, wherein the enzyme that inhibits a macrolide is an erythromycin esterase or an erythromycin phosphotransferase.

6. The hybrid protein molecule according to claim 1, wherein the enzyme that inhibits a tetracycline is an NADPH-dependent oxydoreductase tetracycline.

7. The hybrid protein molecule according to claim 1, wherein the enzyme that inhibits a lincosamide is a lincomycin nucleotidyltransferase.

8. The hybrid protein molecule according to claim 1, wherein at least one of the enzymes comprises a sequence having a sequence homology of at least 40% with SEQ ID No. 1.

9. The hybrid protein molecule according to claim 1, wherein at least one of the enzymes comprises a sequence having a sequence homology of at least 40% with SEQ ID No. 2.

10. The hybrid protein molecule according to claim 1, wherein at least one of the enzymes comprises a sequence having a sequence homology of at least 40% with SEQ ID No. 3.

11. The hybrid protein molecule according to claim 1, wherein at least one of the enzymes comprises a sequence having a sequence homology of at least 40% with SEQ ID No. 4.

12. The hybrid protein molecule according to claim 1, wherein at least one of the enzymes comprises a sequence having a sequence homology of at least 40% with SEQ ID No. 5.

13. The hybrid protein molecule according to claim 1, wherein at least one of the enzymes comprises a sequence having a sequence homology of at least 40% with SEQ ID No. 6.

14. The hybrid protein molecule according to claim 1, wherein at least one of the enzymes comprises a sequence having a sequence homology of at least 40% with SEQ ID No. 7.

15. The hybrid protein molecule according to claim 1, wherein the enzymes are fused into a single stranded protein.

16. The hybrid protein molecule according to claim 1, wherein the enzymes are linked together covalently by cross-linking.

17. A pharmaceutical composition comprising the hybrid protein molecule according to claim 1.

18. (canceled)

19. (canceled)

20. The pharmaceutical composition according to claim 17, wherein said composition is a dosage form for oral administration.

21. The pharmaceutical composition according to claim 17, further comprising at least one gastroprotective agent.

22. A method for the production of the hybrid protein molecule in accordance with claim 1 in a non-human living recombinant organism.

23. A method for reducing an intestinal side effect of at least one antibiotic comprising administering the hybrid protein molecule of claim 1 to a human or animal before, at the same time or after administration of at least one antibiotic.

24. The method of claim 23, wherein the intestinal side effect is a nosocomial infection, diarrhea, or a combination thereof.
